(19) United States
(12) Patent Application Publication
(10) Pub. No.: US 2024/0285586 A1
(43) Pub. Date: Aug. 29, 2024

(54) METHODS FOR TREATING BACTERIAL CELL POPULATIONS

(71) Applicant: THE HONG KONG POLYTECHNIC UNIVERSITY, Hong Kong (CN)

(72) Inventors: Sheng CHEN, Hong Kong (CN); Wai Chi Edward CHAN, Hong Kong (CN); Chen XU, Hong Kong (CN); Miaomiao WANG, Hong Kong (CN)

(21) Appl. No.: 18/568,898

(22) PCT Filed: Jun. 11, 2021
(86) PCT No.: PCT/CN2021/099665

§ 371 Date: Dec. 11, 2023

(51) Int. Cl. A61K 31/4174; A61K 45/06; A61P 31/04

(52) U.S. Cl. CPC A61K 31/4174; A61K 45/06; A61P 31/04

(57) ABSTRACT

Provided herein are methods and compositions useful for treating bacterial cell populations including bacterial persister cells and/or antibiotic resistant bacterial cells.

### TECHNICAL FIELD

The present disclosure generally relates to methods and compositions useful for treating bacterial cell populations. More particularly, the present disclosure provides methods and compositions useful in treating bacterial cell populations comprising bacterial persister cells and/or antibiotic resistant bacterial cells.

### BACKGROUND

Bacterial antibiotic tolerance is loosely defined as a phenomenon in which bacteria always contain a sub-population, which exhibits the ability to withstand the deleterious effects of antibiotics at concentrations that can otherwise be lethal, yet such sub-population can re-grow under favorable conditions and generate antibiotic-susceptible offspring. Recent studies show that re-growth of antibiotic-tolerant cells that reside in the human body for a prolonged period is responsible for causing a wide range of chronic and recurrent infections, especially among immuno-compromised patients. It is known that more than 80% of cystic fibrosis patients would become chronically infected by P. aeruginosa or S. aureus; such infections are often associated with a rapid decline in lung function and a high risk of death. Indwelling devices and catheter infections related to tolerant biofilms formed by S. aureus, P. aeruginosa, S. typhimurium, E. coli and other bacteria account for about half of nosocomial infections, rendering these devices effectively unusable. Bacterial tolerance has been reported in almost all clinically important bacterial pathogens such as P. aeruginosa, A. baumannii, K. pneumoniae, S. typhimurium, S. aureus and E. coli. Complete eradication of bacterial tolerant sub-population needs to be achieved in order to prevent occurrence of chronic and recurrent infections in seriously ill patients. It is also an important step in clinical treatment because a single bacterium that remains can re-grow and cause recurrent infection. Devising a universal approach to completely eradicate antibiotic tolerant sub-population of clinically important bacterial pathogens, would save millions of lives each year.

Complete eradication of tolerant cells is almost impossible by inhibiting just one specific cellular function. Two previous publications reported complete eradication of antibiotic tolerant sub-population in Gram positive bacteria by using the retinoid and acyldepsipeptide antibiotic to inflict membrane damage and activate casein lytic proteases respectively. However, these agents are not effective on Gram negative organisms.

Bacterial persister cells can also exhibit antibiotic resistance owing, at least in part, to their dormant state. Bacterial persister cells that awaken can result in recurrent infection.

There thus exists a need for improved methods and compositions for treating bacterial cell populations comprising antibiotic resistant bacterial cells and/or bacterial persister cells.

### SUMMARY

It was found that active maintenance of bacterial transmembrane proton motive force (PMF) is essential for starvation-induced tolerance in bacteria, and that disruption of PMF resulted in eradication of the entire antibiotic-resistant and/or persister sub-population.

The present disclosure provides a strategy for treating a bacterial cell population comprising antibiotic resistant bacterial cells and/or bacterial persister cells by administering an agent capable of disrupting the bacterial PMF and optionally an antibacterial agent.

In a first aspect provided herein is a method for treating a bacterial infection in a subject in need thereof, the method comprising: administering a therapeutically effective amount of a bacterial transmembrane proton motive force (PMF) inhibitor to the subject, wherein the bacterial infection is the result of a bacterial cell population comprising persister bacterial cells, antibiotic resistant bacterial cells, or a mixture thereof, wherein the PMF inhibitor is an imidazole-based antifungal agent with the proviso that the PMF inhibitor is not 4-(2-amino-1H-imidazol-4-yl)-N-(tridecan-7-yl)butanamide or 4-(2-amino-1H-imidazol-4-yl)-N-tridecylbutanamide.

In certain embodiments, the bacterial cell population is a Gram-negative bacterial cell population.

In certain embodiments, the imidazole-based antifungal agent is selected from the group consisting of clotrimazole, econazole, sertaconazole, sulconazole, tioconazole, luliconazole, isoconazole, miconazole, enilconazole, fenticonazole, ketoconazole, climbazole, butoconazole, oxiconazole, fluconazole, voriconazole, letrozole, triclabendazole, thiabendazole, fenbendazole, and omeprazole or a pharmaceutically acceptable salt thereof.

In certain embodiments, the PMF inhibitor is administered in an amount effective to at least partially inhibit PMF in the bacterial cell population.

In certain embodiments, the bacterial infection is the result of a bacterial cell population consisting of 50% or more of persister bacterial cells, antibiotic resistant bacterial cells, or a mixture thereof.

In certain embodiments, the bacterial infection is the result of a bacterial cell population consisting of 90% or more of persister bacterial cells, antibiotic resistant bacterial cells, or a mixture thereof.

In certain embodiments, the bacterial infection is the result of a bacterial cell population consisting essentially of antibiotic resistant bacteria selected from the group consisting of E. coli, K. pneumoniae, A. baumannii, P. aeruginosa, S. aureus, and S. typhimurium.

In certain embodiments, the method further comprises the step of co-administering a therapeutically effective amount of an antibacterial to the subject.

In certain embodiments, the bacterial infection is the result of a bacterial cell population consisting of 50% or more of persister bacterial cells, antibiotic resistant bacterial cells, or a mixture thereof.

In certain embodiments, the imidazole-based antifungal agent is selected from the group consisting of econazole, sertaconazole, sulconazole, tioconazole, luliconazole, and isoconazole, or a pharmaceutically acceptable salt thereof.

In certain embodiments, the antibacterial is selected from the group consisting of: a β-lactam, an aminoglycoside, a quinolone, a glycopeptide, a glycylcycline, a lipopeptide, a macrolide, chloramphenicol, a dihydrofolate reductase inhibitor, a sulfonamide, rifampicin, metronidazole, clindamycin, linkomycin, fusidic acid, furazolidone, isoniazid, and pyrazinamide.

In certain embodiments, the antibacterial is selected from the group consisting of ampicillin, ceftazidime, ciprofloxacin, gentamycin, meropenem, and colistin or a pharmaceutically acceptable salt thereof.

In certain embodiments, the imidazole-based antifungal agent is selected from the group consisting of econazole, sertaconazole, sulconazole, tioconazole, luliconazole, isoconazole, and miconazole or a pharmaceutically acceptable salt thereof; and the antibacterial is colistin or a pharmaceutically acceptable salt thereof.

In certain embodiments, the imidazole-based antifungal agent is econazole or a pharmaceutically acceptable salt thereof; and the antibacterial is selected from the group consisting of ampicillin, ceftazidime, ciprofloxacin, gentamycin, meropenem, and colistin or a pharmaceutically acceptable salt thereof.

In certain embodiments, the bacterial infection is the result of a bacterial cell population consisting of 90% or more of persister bacterial cells, antibiotic resistant bacterial cells, or a mixture thereof.

In a second aspect, provided herein is a method of re-sensitizing a persister bacterial cell or an antibiotic resistant bacterial cell to an antibacterial, the method comprising: contacting the persister bacterial cell or the antibiotic resistant bacterial cell with a PMF inhibitor, wherein the PMF inhibitor is an imidazole-based antifungal agent with the proviso that the PMF inhibitor is not 4-(2-amino-1H-imidazol-4-yl)-N-(tridecan-7-yl)butanamide or 4-(2-amino-1H-imidazol-4-yl)-N-tridecylbutanamide.

In certain embodiments, the persister bacterial cell or the antibiotic resistant bacterial cell is a Gram-negative persister bacterial cell or Gram-negative antibiotic resistant bacterial cell.

In certain embodiments, the imidazole-based antifungal agent is selected from the group consisting of clotrimazole, econazole, sertaconazole, sulconazole, tioconazole, luliconazole, isoconazole, miconazole, enilonazole, fenticonazole, ketoconazole, climbazole, butoconazole, oxiconazole, fluconazole, voriconazole, letrozole, triclabendazole, thiabendazole, fenbendazole, and omeprazole or a pharmaceutically acceptable salt thereof.

In certain embodiments, the imidazole-based antifungal agent is econazole or a pharmaceutically acceptable salt thereof.

In certain embodiments, the persister bacterial cell or the antibiotic resistant bacterial cell is selected from the group consisting of E. coli, K. pneumoniae, A. baumannii, P. aeruginosa, S. aureus, and S. typhimurium.

### BRIEF DESCRIPTION OF THE DRAWINGS

The above and other objects and features of the present disclosure will become apparent from the following description of the disclosure, when taken in conjunction with the accompanying drawings.

FIG. 1. Activated Psp response during nutrient starvation affects bacterial survival and antibiotic tolerance. (A) The wild type E. coli BW25113 strain and the ΔpspA gene knockout strain were starved for 24 hrs, followed by treatment with ampicillin at 100 μg/mL for 144 hrs, variation in CFU recorded at different time points is shown. P value was tested between ΔpspA and ΔpspA+AMP at 144 hr. (B) Western blot analysis of the PspA protein in bacterial population at a cell density of OD₆₀₀ 0.2 which had been subjected to starvation for 24 hrs, with the endogenous protein GAPDH as control. The relative expression level of the PspA protein recorded in log phase population and bacteria which had experienced nutrient starvation for 24 hours upon normalization with the GAPDH control is shown. Data are the average of at least two independent experiments performed with three biological replicates (n≥6). ** indicate a P value of <0.01, *** indicate a P value of <0.001 by two-tailed Student's test. Error bar represents standard deviation.

FIG. 2. Psp response helps maintain PMF during starvation in E. coli without altering membrane permeability. (A) Fluorescence intensity of SYTOX Green, which was used to detect membrane permeability of the wild type and ΔpspA strains, showed that the cell membrane in the ΔpspA strain remained intact; strains treated with colistin were included as positive control. (B) Comparison between the fluorescence intensity of DiSC₃(5)-stained cells in the exponential growth phase of both wild type and ΔpspA strains reveals the same initial intensity and a similar degree of changes in membrane potential upon addition of valinomycin (labelled as V). P values were tested between WT and ΔpspA, WT+V and ΔpspA+V at the beginning time. (C) Comparison between the fluorescence intensity of DiSC₃(5)-strained wild type and the ΔpspA strain which had been subjected to starvation for 24 hrs depicts a much higher fluorescence intensity and hence a much lower PMF in the ΔpspA mutant. P values were tested between WT and ΔpspA, WT+V and ΔpspA+V at the beginning time. (D) Comparison between the fluorescence intensity of DiSC₃(5)-stained exponentially growing wild type population and those which had been subjected to 24 hrs starvation reveals a similar initial fluorescence intensity and also a similar degree of changes in fluorescence intensity, and hence membrane potential, upon addition of valinomycin. Data are the average of at least two independent experiments performed with three biological replicates (n≥6). Error bar represents standard deviation. P values were tested between log and starvation, log+V and starvation+V at the beginning time. (E) Confocal microscopy images of DiSC₃(5)-stained cells which have been subjected to 24 hrs starvation in the absence and presence of valinomycin. The left and right panels are the fluorescence and bright field images respectively (scale bar: 4 μm). (F) The mean DiSC₃(5) fluorescence intensity of confocal microscopy image, which was calculated by the LAS X software. Data are the average of three observation field images. ns indicate no significance, ** indicate a P value of <0.01, *** indicate a P value of <0.001 by two-tailed Student's test. Error bar represents standard deviation.

FIG. 3. PMF is essential for maintaining the tolerance phenotype in bacteria under starvation. (A) Population size of E. coli strain BW25113 recorded at different time points upon starvation for 24 hrs, followed by treatment with ampicillin, sodium azide, CCCP or various combinations of such compounds. P values were tested between WT+NaN3 and WT+NaN3+AMP, WT+CCCP and WT+CCCP+AMP at 144 hr. (B) Population size of the ΔpspA gene knockout mutant recorded at different treatment time points upon starvation for 24 hrs and then treatment with ampicillin, sodium azide, CCCP or various combinations of such compounds. P values were tested between ΔpspA+NaN3 and ΔpspA+NaN3+AMP, ΔpspA+CCCP and ΔpspA+CCCP+AMP at indicated time points. (C) E. coli strain BW25113 and the corresponding Δndh and ΔnuoI gene knockout mutants and double gene knockout mutant ΔndhΔnuoI (D) were starved for 24 hrs, followed by treatment with ampicillin for 144 hrs. Changes in population size during the course of 144 hrs are shown, along with data recorded in a no ampicillin-treatment control. Data are the average of at least two independent experiments performed with three biological replicates (n≥6). ns indicates no significance, ** indicate a P value of <0.01, *** indicate a P value of <0.001 by two-tailed Student's test. Error bar represents standard deviation.

FIG. 4. Active efflux driven by PMF contributes partially to formation of an antibiotic tolerant sub-population during starvation. (A-D) Fluorescence intensity recorded by flow cytometry depicts the degree of antibiotic accumulation (BOCILLIN™ FL Penicillin, 10 μg/mL) in wild type or ΔpspA subjected to 24 hrs starvation in the presence and absence of CCCP. P2 gate indicates the population whose BOCILLIN fluorescent intensity is more than 10³ RFU. (E) The fluorescent efflux substrate Nile Red was used to stain wild type bacterial population which had been subjected to 24 hrs starvation in the presence and absence of CCCP. Data are the average of at least two independent experiments performed with three biological replicates (n≥6). Error bar represents standard deviation. (F) Variation in the population size of E. coli strain BW25113 and the ΔtolC gene knockout strain which had been subjected to starvation for 24 hrs, followed by treatment with ampicillin for 144 hrs. A no ampicillin-treatment control of each of the BW25113 and ΔtolC gene knockout strain was included. The effect of the efflux pump inhibitor PAβN on starvation-induced ampicillin tolerance of the BW25113 strain is also depicted. Data are the average of at least two independent experiments, each performed with three biological replicates (n≥6). P values were tested between WT and PAβN+AMP, ΔtolC and ΔtolC+AMP at 144 hr. ** indicate a P value of <0.01, *** indicate a P value of <0.001 by two-tailed Student's test. Error bar represents standard deviation.

FIG. 5. PMF maintenance is essential for starvation-induced tolerance formation in major Gram-negative and Gram-positive bacteria. Changes in the size of antibiotic-tolerant sub-population in P. aeruginosa (A), K. pneumoniae (B), S. aureus (C), A. baumannii (D) and S. typhimurium (E) which had been starved for 24 hrs, followed by treatment with 10×MIC ampicillin (AMP) alone (FIG. 13), CCCP alone and CCCP in the presence of 10×MIC ampicillin. CCCP(100), 100 μM CCCP; CCCP(50), 50 μM CCCP; CCCP(10), 10 μM CCCP; CCCP(5), 5 μM CCCP; CCCP(1), 1 μM CCCP. Data are the average of at least two independent experiments performed with three biological replicates (n≥6). P values were tested between CCCP+AMP and CCCP with the same concentration at indicated time points. ** indicate a P value of <0.01, **** indicate a P value of <0.0001 by two-tailed Student's test. Error bar represents standard deviation.

FIG. 6. Proposed model of PMF-mediated development of starvation-induced tolerance. (A) Maintaining PMF is essential for prolonged survival of starvation-induced tolerant cells. Efflux activities driven by PMF extrude β-lactams to facilitate tolerance formation; other membrane protein activities which presumably involve import/export of specific metabolites/nutrients are supported by PMF and are also important for maintaining a tolerance phenotype. (B) Effect of PMF dissipators such as CCCP on tolerant cell killing. PMF dissipator causes dissipation of bacterial membrane PMF and hence inhibition of ATP production, which in turn affects a series of cellular functions that are involved in maintaining the tolerance phenotype, leading to killing of tolerant cells. (C) Effect of PMF dissipator and ampicillin on tolerant cell killing. Tolerant cells are eradicated more effectively in the presence of β-lactam if PMF cannot be maintained under starvation stress. Dissipation of bacterial membrane PMF inactivates antibiotic efflux activities, leading to accumulation of antibiotic in the periplasm of tolerant cells. The accumulation of antibiotics and arrest of other cellular functions lead to more effective killing of tolerant cells. (D) Dissipation of PMF with or without the presence of antibiotic could both lead to tolerant cells killing. The cellular basis of re-sensitization of tolerant cells to β-lactams upon PMF dissipation remains to be elucidated.

FIG. 7. Bacterial antibiotic tolerance was negatively affected by pspA deletion. (A) Relative tolerance ratio of the wild type strain and psp mutants calculated by comparing the size of bacterial population that survived treatment with ampicillin at 100 μg/mL for 144 hrs upon starvation for 24 hrs to those without ampicillin treatment. (B) Complementation of ΔpspA with plasmid-borne copies of pspA restored tolerance to ampicillin. Wild type and ΔpspA are included as control. P value was tested between ΔpspA and ΔpspA+AMP at 144 hr. (C) The size of population of wild type and ΔpspA upon starvation for 24 hrs followed by treatment with 10 μg/mL gentamicin (Gen) or 0.5 μg/mL ciprofloxacin (Cip) for 144 hrs. Data are the average of at least two independent experiments performed with three biological replicates (n≥6). P value was tested between WT+Gen and ΔpspA+Gen at 144 hr. * indicate a P value of <0.05, *** indicate a P value of <0.001 by two-tailed Student's test. Error bar represents standard deviation.

FIG. 8. Assessment of antibiotic susceptibility of bacterial sub-population exhibiting starvation-induced antibiotic tolerance. In order to confirm that the antibiotic tolerance phenotype observed in the tolerance assay was not due to existence of a resistant sub-population, bacteria subjected to nutrient starvation for 24 hrs were split into two portions, one was treated with 100 μg/mL ampicillin for 4 hrs to obtain antibiotic tolerant sub-population, and the one without antibiotic was set as control. The tolerant sub-population was then collected by centrifugation, followed by re-suspension and dilution in fresh LB and incubation at 37° C. to induce regrowth. Fresh bacterial culture derived from this tolerant sub-population was subjected to antibiotic susceptibility tests, with results confirming that offspring of such sub-population remained susceptible to the test agent. Two biological replicates were tested.

FIG. 9. PMF dissipation negatively affected bacterial tolerance to ampicillin and gentamycin, but not ciprofloxacin. (A) The size of the bacterial population of the wild type E. coli strain BW25113 that survived at different time points upon starvation for 24 hrs, followed by treatment with ampicillin, CCCP or a combination of these two compounds. CCCP (100), 100 μM CCCP; CCCP (10), 10 μM CCCP; CCCP (1), 1 μM CCCP; CCCP (0.1), 0.1 μM CCCP. P values were tested between CCCP and CCCP+AMP with the same concentration. (B-E) The size of the bacterial population of the wild type or ΔpspA strains that survived at different time points upon starvation for 24 hrs, followed by treatment with gentamicin (10 μg/mL), ciprofloxacin (0.5 μg/mL), CCCP (1 μM) or a combination of such compounds. P values were tested between Gen/Cip and CCCP+Gen/Cip at indicated time points. (F) Complementation of ΔndhΔnuoI with plasmid-borne copies of ndh and nuoI restored tolerance to ampicillin. Wild type and ΔndhΔnuoI are included as control. P values were tested between ΔndhΔnuoI and ΔndhnuoI+AMP, empty vector and empty vector+AMP at indicated time points. ns indicates no significance, * indicates a P value of <0.05, ** indicates a P value of <0.01, *** indicates a P value of <0.001, **** indicates a P value of <0.0001 by two-tailed Student's test. Error bar represents standard deviation.

FIG. 10. Evaluation of intracellular fluorescent β-lactam amount and the effect of efflux pumps upon bacterial tolerance. (A) CCCP does not affect the level of fluorescence exhibited by BOCILLIN. The fluorescence signal of bacterial population treated with CCCP only (no BOCILLIN) was measured and compared with those treated with both CCCP and BOCILLIN (with BOCILLIN). (B-F) FSC-SSC profiles of BOCILLIN stained wild type and ΔpspA cells with or without CCCP(1 μM). P1 gate was determined as the bacteria sector since the percentage of P1 in samples (˜30%) is much higher than that in water (˜2%). (G) Complementation of ΔtolC with plasmid-borne copies of tolC restored tolerance to ampicillin. Wild type and ΔtolC were included as control. P value was tested between ΔtolC and ΔtolC+AMP at 144 hr. (H) Growth rate of wild type strain in the presence and absence of PAβN (100 μM). The results show that PAβN does not inhibit bacterial growth. ns indicates no significance, **** indicates a P value of <0.0001 by two-tailed Student's test. Error bar represents standard deviation.

FIG. 11: A total of 58 shortlisted genes whose expression level was found to be up-regulated by three folds or more in RNA-Seq upon starvation for 24 hrs. *Fold difference in expression level of the test genes in E. coli population which had been starved for 24 hrs, with exponentially growing population of identical cell density as control. 1 Efflux and membrane protein genes. 2 Transcriptional regulator genes. 3 Envelope stress and chaperone genes.⁴ Oxidative enzyme genes.⁵ DNA repair genes.⁶ Starvation stress sensing genes.

FIG. 12. MIC of gene knockout strains

FIG. 13. MIC of ampicillin for bacterial strains of various species.

FIG. 14. E. coli strains used in the examples. *Baba, T., et al., Construction of Escherichia coli K-12 in-frame, single-gene knockout mutants: the Keio collection. Mol Syst Biol, 2006. 2: p. 2006 0008.

FIG. 15. Minimal inhibitory concentration (MIC) of ampicillin and econazole tested against bacterial strains of various species.

FIG. 16. Econazole kills persisters of major Gram-negative and Gram-positive bacterial pathogens through dissipation of proton motive force (PMF). Changes in the size of antibiotic-tolerant sub-population in E. coli (A), K. pneumoniae (B), A. baumannii (C), P. aeruginosa (D), S. aureus (E) and S. typhimurium (F) which had been starved for 24 hrs, followed by treatment with ampicillin (AMP, 10×MIC) alone, econazole (Econ) alone, ceftazidime (CAZ, 100 μg/ml) alone (in S. typhimurium), and econazole in combination with ampicillin and ceftazidime. (G) Changes in the size of antibiotic-tolerant sub-population in E. coli, which had been starved for 24 hrs, followed by treatment with ampicillin alone, CCCP alone, econazole alone and the combination of ampicillin and econazole or CCCP. (H) Changes in the size of antibiotic-tolerant sub-population in E. coli, which had been starved for 24 hrs, followed by treatment with ciprofloxacin (CIP, 1 μg/ml) or gentamycin (GEN, 20 μg/ml) alone or combination with econazole (Econ). (I) Changes in the size of antibiotic-tolerant sub-population in carbapenem-resistant E. coli (bla_{NDM-1}-bearing E. coli J53), which had been starved for 24 hrs, followed by treatment with meropenem (Mer, 40 μg/ml) alone, econazole alone or combination of both. Data are the average of at least two independent experiments performed with three biological replicates (n≥6).

FIG. 17. Econazole (Econ) causes dissipation of bacterial PMF. Fluorescence intensity of DiSC₃(5), which measures membrane potential of bacteria, was found to increase significantly over a period of 10 min time after treatment with Econ in E. coli (A), S. aureus (B) and P. aeruginosa (C) when compared to the no-treatment control, suggesting that Econ could cause dissipation of membrane PMF in these organisms.

FIG. 18. SEM images of E. coli cells treated with econazole, ampicillin and a combination of both. (A) Intact membrane and intracellular contents were visible in cells without any treatment; (B, C) upon exposure to ampicillin (100 μg/ml and 1000 μg/ml), smooth surface and intracellular content could still be seen, but there was slight shrinkage at one pole of the cell (arrow 1). (D) Treatment with 40 μM econazole (Econ) alone resulted in severe structural damages in cellular membrane and cytosol leakage (arrow 2). (E) Treatment with econazole (40 μM) and ampicillin (100 μg/ml) caused cell lysis and almost total loss of the content of cytosol (arrow 3). Arrows depict areas where cell membrane was damaged.

FIG. 19. Ceftazidime and econazole combination therapy could effectively eradicate bacterial tolerant cells in in vivo mouse model. (A) E. coli BW25113 mice deep-seated thigh infection model. 1×10⁶ CFU of E. coli BW25113 were injected into the right thigh of the test animal. At 24 hrs post-infection, the mice were subjected to the indicated antibacterial treatment (i.p.) every 12 hrs for 72 h. The mice were euthanized and the infected thighs were aseptically excised, homogenized in PBS, followed by determination of the bacterial load. (B) S. typhimurium PY1 tolerance sepsis model. The mice were intraperitoneally injected with 7.6×10⁵ CFU S. typhimurium PY01. After 24 h, the mice were subjected to indicated therapies (i.p.) every 12 h. The mortality rate of the test mice was recorded for 72 h. (C) Mice that survived in (B) were euthanized, peritoneal washes were performed by injection 2 mL of saline into the intraperitoneal space, followed by massage of the abdomen. The abdomen was then cut open and 200 μL of peritoneal fluid was collected for determination of bacterial cell count. (D) S. typhimurium PY1 tolerance sepsis model; same as (B) with the only difference being inoculation with a higher amount of S. typhimurium (1.5×10⁶ CFU). (E) Since most of the test animals were dead, bacterial survival assay was not performed. In the same tolerance sepsis model, a lower dose of 2.8×10¹ CFU of S. typhimurium PY01 was used. After 24 h, the mice were subjected to indicated therapies (i.p.) every 12 h. The mortality rate of mice was recorded for 72 h. (F) Mice that survived in (E) were euthanized, peritoneal washes were performed by injection of 2 mL of saline in the intraperitoneal space followed by massage of the abdomen. The abdomen was then cut open and 200 μL of peritoneal fluid were collected for determination of bacterial count. Mice treated with ceftazidime (CAZ) (20 mg/kg) only exhibited significantly slower (P=0.0004) rate of eradication of S. typhimurium PY1 tolerant sub-population when compared to treatment with the econazole (Econ) and ceftazidime combination (20 mg/kg). Econ20, econazole (20 mg/kg); CAZ20, ceftazidime (20 mg/kg). One-way ANOVA and post hoc Tukey test was used. *P<0.05; ****P<0.0001.

FIG. 20. Shows the result of experiments in which Compound No. 1-23 and colistin were tested alone and in combination in E. coli J53 (mcr-1). The MICs of colistin in the presence and absence of Compound No. 1-23 against colistin-resistant E. coli was determined using broth dilution method according to the CLSI criteria of 2016.

FIG. 21. Shows the chemical structures of Compound No. 1-23.

### DETAILED DESCRIPTION

### Definitions

Throughout the application, where compositions are described as having, including, or comprising specific components, or where processes are described as having, including, or comprising specific process steps, it is contemplated that compositions of the present teachings can also consist essentially of, or consist of, the recited components, and that the processes of the present teachings can also consist essentially of, or consist of, the recited process steps.

In the application, where an element or component is said to be included in and/or selected from a list of recited elements or components, it should be understood that the element or component can be any one of the recited elements or components, or the element or component can be selected from a group consisting of two or more of the recited elements or components. Further, it should be understood that elements and/or features of a composition or a method described herein can be combined in a variety of ways without departing from the spirit and scope of the present teachings, whether explicit or implicit herein.

It should be understood that the order of steps or order for performing certain actions is immaterial so long as the present teachings remain operable. Moreover, two or more steps or actions may be conducted simultaneously.

The use of the singular herein includes the plural (and vice versa) unless specifically stated otherwise. In addition, where the use of the term “about” is before a quantitative value, the present teachings also include the specific quantitative value itself, unless specifically stated otherwise. As used herein, the term “about” refers to a ±10%, ±7%, ±5%, ±3%, ±1%, or ±0% variation from the nominal value unless otherwise indicated or inferred.

The term “Gram-positive bacteria” as used herein refers to bacteria characterized by having as part of their cell wall structure peptidoglycans as well as polysaccharides and/or teichoic acids and are characterized by their blue-violet color reaction in the Gram-staining procedure.

The term “Gram-negative bacteria” as used herein refers to bacteria characterized by the presence of a double membrane surrounding each bacterial cell and are characterized by the absence of color upon washing out with a decolorizer and counter-staining pink with safranin in the Gram-staining procedure.

The term “composition” is intended to encompass a product comprising the specified ingredients in the specified amounts, as well as any product that results, directly or indirectly, from combinations of the specified ingredients in the specified amounts.

As used herein, unless otherwise indicated, the term “treating” means reversing, alleviating, inhibiting the progress of, or preventing the disorder or condition to which such term applies, or one or more symptoms of such disorder or condition. The term “treatment”, as used herein, refers to the act of treating, as “treating” is defined immediately above.

The term “subject” as used herein, refers to an animal, typically a mammal or a human, that will be or has been the object of treatment, observation, and/or experiment. When the term is used in conjunction with administration of a compound described herein, then the subject has been the object of treatment, observation, and/or administration of the compound described herein.

The terms “co-administration” and “co-administering” refer to both concurrent administration (administration of two or more therapeutic agents at the same time) and time varied administration (administration of one or more therapeutic agents at a time different from that of the administration of an additional therapeutic agent or agents), as long as the therapeutic agents are present in the patient to some extent at the same time.

The term “therapeutically effective amount” as used herein, means that amount of active compound or pharmaceutical agent that elicits a biological, medicinal, or imaging response in a cell culture, tissue system, subject, animal, or human that is being sought by a researcher, veterinarian, clinician, or physician, which includes alleviation of the symptoms of the disease, condition, or disorder being treated and/or achieving the desired degree of magnetic resonance imaging contrast enhancement.

As used herein, unless otherwise indicated, the phrase “pharmaceutically acceptable salt(s)” includes salts of acidic or basic groups which may be present in the compounds described herein. The compounds described herein that contain basic groups, such as amines, are capable of forming a wide variety of salts with various inorganic and organic acids. The acids that may be used to prepare pharmaceutically acceptable acid addition salts of such basic compounds described herein are those that form relatively non-toxic acid addition salts, i.e., salts containing pharmacologically acceptable anions, such as acetate, benzenesulfonate, benzoate, bicarbonate, bisulfate, bitartrate, borate, bromide, calcium edetate, camsylate, carbonate, chloride, clavulanate, citrate, dihydrochloride, edetate, edislyate, estolate, esylate, ethylsuccinate, fumarate, gluceptate, gluconate, glutamate, glycollylarsanilate, hexylresorcinate, hydrabamine, hydrobromide, hydrochloride, iodide, isothionate, lactate, lactobionate, laurate, malate, maleate, mandelate, mesylate, methylsulfate, mucate, napsylate, nitrate, oleate, oxalate, pamoate (embonate), palmitate, pantothenate, phosphate/diphosphate, polygalacturonate, salicylate, stearate, subacetate, succinate, tannate, tartrate, teoclate, tosylate, triethiodode, and valerate salts.

In other cases, the compounds described herein may contain one or more acidic functional groups and, thus, are capable of forming pharmaceutically-acceptable salts with pharmaceutically-acceptable bases. The term “pharmaceutically-acceptable salts” in these instances refers to the relatively non-toxic, inorganic and organic base addition salts of compounds of the present invention. These salts can likewise be prepared in situ in the administration vehicle or the dosage form manufacturing process, or by separately reacting the purified compound in its free acid form with a suitable base, such as the hydroxide, carbonate or bicarbonate of a pharmaceutically-acceptable metal cation, with ammonia, or with a pharmaceutically-acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like.

The present disclosure provides a method for treating a bacterial infection in a subject in need thereof, the method comprising: administering a therapeutically effective amount of a bacterial transmembrane proton motive force (PMF) inhibitor to the subject, wherein the bacterial infection is the result of a bacterial cell population comprising persister bacterial cells, antibiotic resistant bacterial cells, or a mixture thereof, wherein the PMF inhibitor is an imidazole-based antifungal agent with the proviso that the PMF inhibitor is not 4-(2-amino-1H-imidazol-4-yl)-N-(tridecan-7-yl)butanamide or 4-(2-amino-1H-imidazol-4-yl)-N-tridecylbutanamide as shown below:

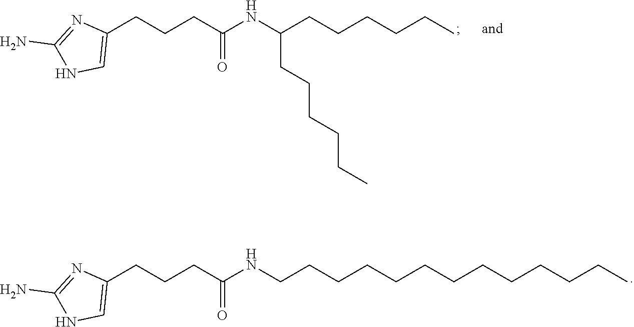 [SMILES: CCCCCCC(CCCCCC)NC(=O)CCCc1c[nH]c(N)n1 ; CCCCCCCCCCCCCNC(=O)CCCc1c[nH]c(N)n1]

In certain embodiments, the subject a canine, feline, bovine, equine, non-human primate, or human. In certain embodiments, the subject is a human.

The imidazole-based antifungal agent can be selected from the group consisting of arasertaconazole, bifonazole, clotrimazole, croconazole, eberconazole, econazole, neticonazole, sertaconazole, sulconazole, tioconazole, luliconazole, isoconazole, miconazole, enilonazole, fenticonazole, ketoconazole, climbazole, butoconazole, oxiconazole, fluconazole, voriconazole, letrozole, triclabendazole, thiabendazole, fenbendazole, and omeprazole or a pharmaceutically acceptable salt thereof. In certain embodiments, the imidazole-based antifungal agent can be selected from the group consisting econazole, sertaonazole, sulonazole, tioonazole, lulionazole, isoconazole, mionazole, and nilonazole. In certain embodiments, the imidazole-based antifungal agent is econazole.

The methods described herein are useful for treating any bacterial infection caused by a population of bacterial cells comprising persister bacterial cells, antibiotic resistant bacterial cells, or a mixture thereof. The methods described herein can be bactericidal or bacteriostatic. In certain embodiments, the methods are bactericidal.

The bacteria can be Gram-positive bacteria, Gram-negative bacteria, Gram-variable bacteria, or Gram-indeterminate bacteria.

Exemplary Gram-negative bacteria include, but are not limited to, Acinetobacter calcoaceticus, Acinetobacter baumannii, Actinobacillus actinomycetemcomitans, Aeromonas hydrophila, Alcaligenes xylosoxidans, Bacteroides, Bacteroides fragilis, Bartonella bacilliformis, Bordetella spp., Borrelia burgdorferi, Branhamella catarrhalis, Brucella spp., Campylobacter spp., Chlamydia pneumoniae, Chlamydia psittaci, Chlamydia trachomatis, Chromobacterium violaceum, Citrobacter spp., Eikenella corrodens, Enterobacter aerogenes, E. coli, Flavobacterium meningosepticum, Fusobacterium spp., Haemophilus influenzae, Haemophilus spp., Helicobacter pylori, Klebsiella pneumoniae, Klebsiella spp., Legionella spp., Leptospira spp., Moraxella catarrhalis, Morganella morganii, Mycoplasma pneumoniae, Neisseria gonorrhoeae, Neisseria meningitidis, Pasteurella multocida, Plesiomonas shigelloides, Prevotella spp., Proteus spp., Providencia rettgeri, Pseudomonas aeruginosa, Pseudomonas spp., Rickettsia prowazekii, Rickettsia rickettsii, Rochalimaea spp., Salmonella spp., Salmonella typhimurium, Serratia marcescens, Shigella spp., Shigella sonnei, Treponema carateum, Treponema pallidum, Treponema pallidum endemicum, Treponema pertenue, Veillonella spp., Vibrio cholerae, Vibrio vulnificus, and Yersinia enterocolitica, Yersinia pestis.

Exemplary Gram-positive bacteria include, but are not limited to, Actinomyces spp., Bacillus anthracis, Bifidobacterium spp., Clostridium botulinum, Clostridium perfringens, Clostridium spp., Clostridium tetani, Corynebacterium diphtheriae, Corynebacterium jeikeium, Enterococcus faecalis, Enterococcus faecium, Erysipelothrix rhusiopathiae, Eubacterium spp., Gardnerella vaginalis, Gemella morbillorum, Leuconostoc spp., Mycobacterium abscessus, Mycobacterium avium complex, Mycobacterium chelonae, Mycobacterium fortuitum, Mycobacterium haemophilium, Mycobacterium kansasii, Mycobacterium leprae, Mycobacterium marinum, Mycobacterium scrofulaceum, Mycobacterium smegmatis, Mycobacterium terrae, Mycobacterium tuberculosis, Mycobacterium ulcerans, Nocardia spp., Peptococcus niger, Peptostreptococcus spp., Proprionibacterium spp., Sarcina lutea, Staphylococcus aureus, Staphylococcus auricularis, Staphylococcus capitis, Staphylococcus cohnii, Staphylococcus epidermidis, Staphylococcus haemolyticus, Staphylococcus hominis, Staphylococcus lugdanensis, Staphylococcus saccharolyticus, Staphylococcus saprophyticus, Staphylococcus schleiferi, Staphylococcus similans, Staphylococcus warneri, Staphylococcus xylosus, Streptococcus agalactiae (group B streptococcus), Streptococcus anginosus, Streptococcus bovis, Streptococcus canis, Streptococcus equi, Streptococcus milleri, Streptococcus mitior, Streptococcus mutans, Streptococcus pneumoniae, Streptococcus pyogenes (group A streptococcus), and Streptococcus salivarius, Streptococcus sanguis.

In certain embodiments, the bacterial infection is caused by E. coli, K. pneumoniae, A. baumannii, P. aeruginosa, S. aureus, and S. typhimurium.

The bacterial infection can be the result of a bacterial cell population consisting of 5%, 10%, 20%, 30%, 40%, 50%, 60%, 70%, 80%, 90%, 95%, 97%, 99% or more of persister bacterial cells, antibiotic resistant bacterial cells, or a mixture thereof.

The antibiotic resistant bacterial cells may comprise one or more genes that confers resistance to antibiotics. Exemplary genes that can confer at least some degree of antibiotic resistance include, but are not limited to, a beta-lactamase gene, such as bla_{CMY}, bla_{CTX-M}, bla_{OXA}, bla_{IMP}, bla_{VIM}, bla_{DHA}, bla_{KPC}, bla_{MOX}, bla_{ACC}, bla_{FOX}, bla_{EBC}, bla_{NDM}, bla_{TEM}, and bla_{SHV}; a plasmid-mediated mcr gene leading to colistin resistance, such as mcr-1, mcr-1.2, mcr-1.3, mcr-1.4, mcr-1.5, mcr-1.6, mcr-1.7, mcr-1.8, mcr-1.9, mcr-2, mcr-3, mcr-4, mcr-5, mcr-6, mcr-7, mcr-8, mcr-9, mcr-10; a chromosomal mutation leading to colistin resistance, such as pmrA pmrB, phoP/phoQ, arnBCADTEF, mgrB, ramA, crrB; a tetracycline resistance gene, such as tetA and tetR; and an aminoglycoside resistance gene, such as aac, ant, or aph.

In certain embodiments, the method for treating a bacterial infection in a subject in need thereof further comprises the step of co-administering a therapeutically effective amount of an antibacterial or a pharmaceutically acceptable salt thereof to the subject.

The antibacterial can be a β-lactam, an aminoglycoside, a quinolone, a glycopeptide, a glycylcycline, a lipopeptide, a macrolide, chloramphenicol, a dihydrofolate reductase inhibitor, a sulfonamide, rifampicin, metronidazole, clindamycin, linkomycin, fusidic acid, furazolidone, isoniazid, pyrazinamide, an antimicrobial peptide or a combination thereof.

Polymyxins useful in the methods described herein include, but are not limited to, polymyxin A, polymyxin B, polymyxin C, polymyxin D, polymyxin E, and polymyxin A. The polymyxin can also be a polymyxin analog. In such instances, the polymyxin analog can be, for example, the polymyxin analogs described in publications WO 2015/149131, WO 2015/135976, US 2015/0031602, WO 2014/188178, WO 2014/108469, US 2014/0162937, WO 2013/072695, WO 2012/168820, WO 2012051663, US 2012/0283176, US 2010/0160215, US 2009/0215677, WO 2008/017734, U.S. Pat. Nos. 6,380,356, and 3,450,687, the contents of which are hereby incorporated by reference.

In certain embodiments, the polymyxin is colistin A (polymyxin E1) or colistin B (polymyxin E2). In certain embodiments, the colistin A is colistin A sulfate or colistimethate A sodium.

Some examples of beta-lactam antibiotics that can be used in combination with the methods of the present disclosure include, in general beta-lactams comprising penam, carbapenam, oxapenam, penem, carbapenem, monobactam, cephem, carbacephem, or oxacephem cores as shown below.

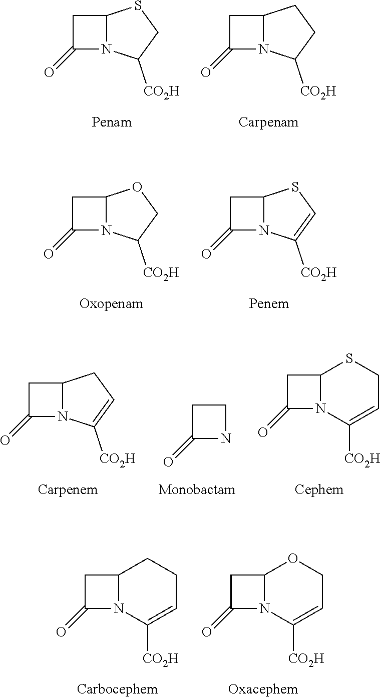

Particularly useful members of those classes include, for example, penams, such as Benzylpenicillin (G), Benzathine Benzylpenicillin, Procaine Benzylpenicillin, Phenoxymethylpenicillin (V), Propicillin, Pheneticillin, Pzidocillin, Plometocillin, Penamecilli, Cloxacillin, Dicloxacillin, Flucloxacillin, Oxacillin, Nafcillin, Methicillin, Amoxicillin, Ampicilli, Pivampicillin, Hetacillin, Bacampicillin, Metampicillin, Talampicillin, Epicillin, Ticarcillin Carbenicillin, Carindacillin, Temocillin, Piperacillin, Azlocillin, Mezlocillin, Mecillinam, Pivmecillinam, and Sulbenicillin, penems, such as Faropenem and Ritipenem, carbapenem, such as Ertapenem, Doripenem, Imipenem, Meropenem, Biapenem, and Panipenem, Cephems, such as Cefazoli, Cefalexin, Cefadroxil, Cefapirin, Cefazedone, Cefazaflur, Cefradine, Cefroxadine, Ceftezole, Cefaloglycin, Cefacetrile, Cefalonium, Cefaloridine, Cefalotin, Cefatrizine, Cefaclor, Cefotetan, Cephamycin, Cefoxitin, Cefprozil, Cefuroxime, Cefuroxime axetil, Cefamandole, Cefminox, Cefonicid, Ceforanide, Cefotiam, Cefbuperazone, Cefuzonam, Cefmetazole, Carbacephem, Loracarbef, Cefixime, Ceftriaxon, Ceftazidime, Cefoperazone, Cefdinir, Cefcapene, Cefdaloxime, Ceftizoxime, Cefmenoxime, Cefotaxime, Cefpiramide, Cefpodoxime, Ceftibuten, Cefditoren, Cefetamet, Cefodizime, Cefpimizole, Cefsulodin, Cefteram, Ceftiolene, Oxacephem, Flomoxef, Latamoxef, Cefepime, Cefozopran, Cefpirome, Cefquinome, Ceftaroline fosamil, Ceftolozane, Ceftobiprole, Ceftiofur, Cefquinome, and Cefovecin, and monobactams, such as Aztreonam Tigemonam, Carumonam, and Nocardicin A.

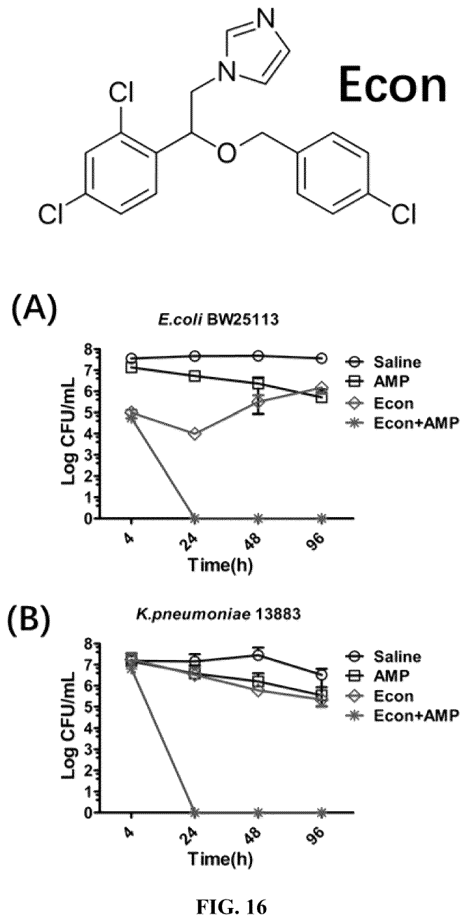

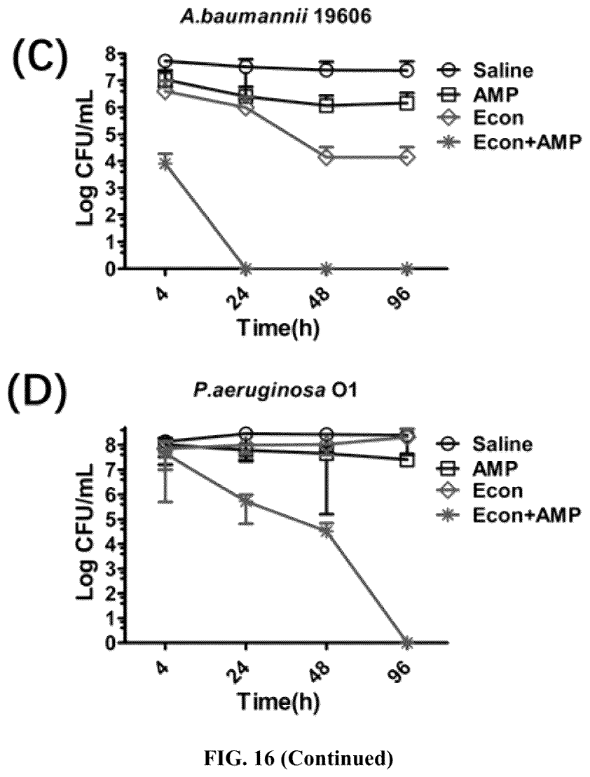

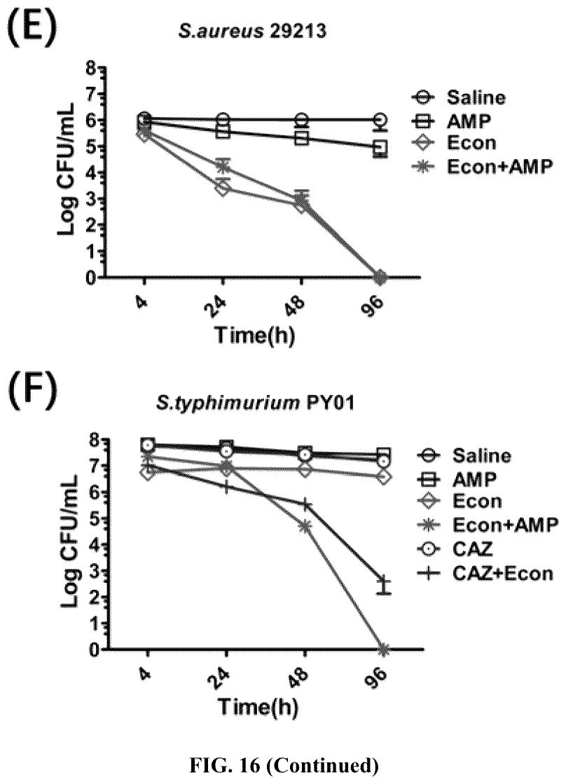

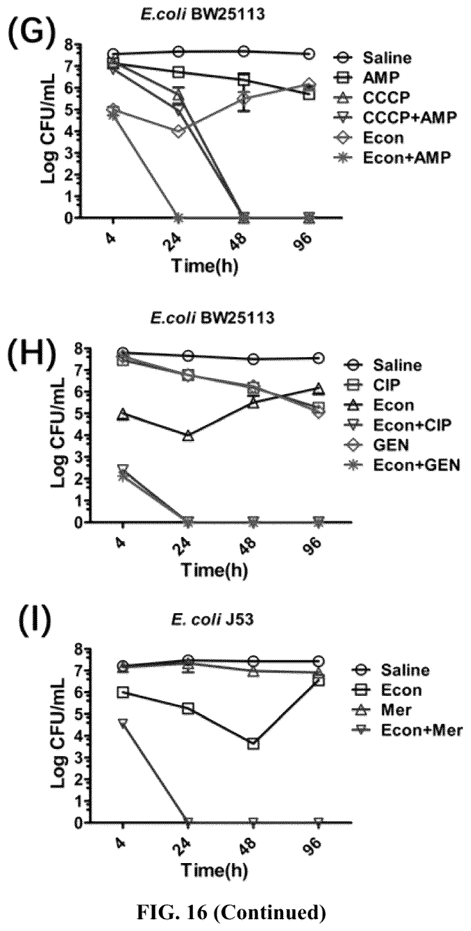

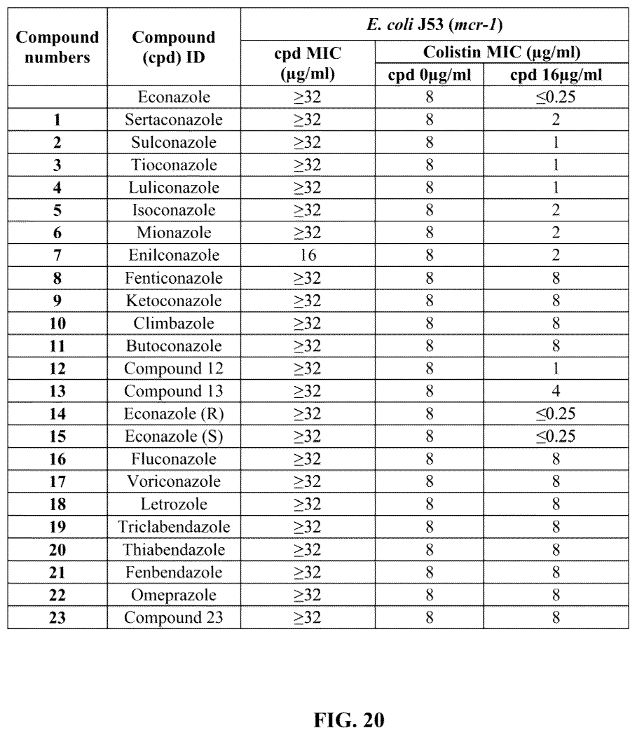

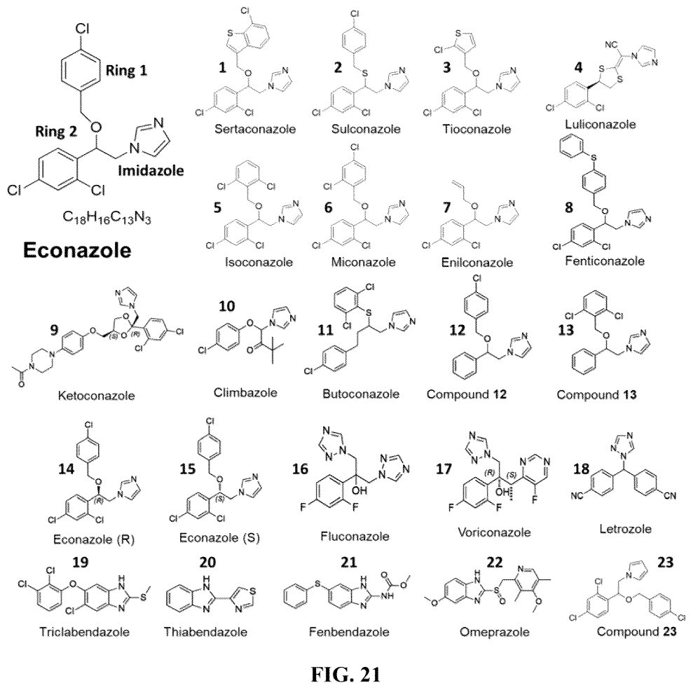

It has been surprisingly discovered that when an imidazole-based antifungal agent is co-administered with an antibacterial to a bacterial cell population comprising persister bacterial cells, antibiotic resistant bacterial cells, or a mixture thereof that a bactericidal synergistic effect is observed. FIG. 16 demonstrates that when econazole is co-administered with the β-lactam, ampicillin, a pronounced bactericidal synergistic effect is observed in the treatment of E. col, S. aureus, K. pneumoniae, A. baumannii, P. aeruginosa, and S. typhimurium. FIG. 20 shows that when econazole, sertaconazole, sulconazole, tioonazole, lulionazole, isoconazole, miconazole, and enilconazole are co-administered with colistin that a bactericidal synergistic effect is observed in the treatment of antibiotic resistant E. coli.

The imidazole-based antifungal agent can be administered according to therapeutic protocols well known in the art. It will be apparent to those skilled in the art that the administration of the imidazole-based antifungal agent and the antibacterial can be varied depending on the disease being treated and the known effects of the antibacterial on that disease. Also, in accordance with the knowledge of the skilled clinician, the therapeutic protocols (e.g., dosage amounts and times of administration) can be varied in view of the observed effects of the administered therapeutic agents (i.e., imidazole-based antifungal agent and antibacterial) on the patient, and in view of the observed responses of the disease to the administered therapeutic agents.

Also, in general, the imidazole-based antifungal agent and the antibacterial do not have to be administered in the same pharmaceutical composition, and may, because of different physical and chemical characteristics, have to be administered by different routes. For example, imidazole-based antifungal agent may be administered intravenously to generate and maintain good blood levels, while the antibacterial may be administered orally. The determination of the mode of administration and the advisability of administration, where possible, in the same pharmaceutical composition, is well within the knowledge of the skilled clinician. The initial administration can be made according to established protocols known in the art, and then, based upon the observed effects, the dosage, modes of administration and times of administration can be modified by the skilled clinician.

The particular choice of antibacterial will depend upon the diagnosis of the attending physicians and their judgment of the condition of the patient and the appropriate treatment protocol.

An imidazole-based antifungal agent and antibacterial may be administered concurrently (e.g., simultaneously, essentially simultaneously or within the same treatment protocol) or sequentially, depending upon the nature of the bacterial infection, the condition of the patient, and the actual choice of antibacterial to be administered in conjunction (i.e., within a single treatment protocol) with the imidazole-based antifungal agent.

If an imidazole-based antifungal agent and the antibacterial are not administered simultaneously or essentially simultaneously, then the optimum order of administration of the imidazole-based antifungal agent and the antibacterial may be different for different bacterial infections. Thus, in certain situations the imidazole-based antifungal agent may be administered first followed by the administration of the antibacterial; and in other situations the antibacterial may be administered first followed by the administration of the imidazole-based antifungal agent. This alternate administration may be repeated during a single treatment protocol. The determination of the order of administration, and the number of repetitions of administration of each therapeutic agent during a treatment protocol, is well within the knowledge of the skilled physician after evaluation of the disease being treated and the condition of the patient. For example, the antibacterial may be administered first and then the treatment continued with the administration of the imidazole-based antifungal agent followed, where determined advantageous, by the administration of the antibacterial, and so on until the treatment protocol is complete.

Thus, in accordance with experience and knowledge, the practicing physician can modify each protocol for the administration of a component (imidazole-based antifungal agent and antibacterial) of the treatment according to the individual patient's needs, as the treatment proceeds.

In certain embodiments, the imidazole-based antifungal agent and the antibacterial are administered sequentially, wherein the antibacterial is administered first and then the imidazole-based antifungal agent is administered alone or in combination with the antibacterial.

The present disclosure also provides a method of re-sensitizing a persister bacterial cell or an antibiotic resistant bacterial cell to an antibacterial, the method comprising: contacting the persister bacterial cell or the antibiotic resistant bacterial cell with a PMF inhibitor, wherein the PMF inhibitor is an imidazole-based antifungal agent with the proviso that the PMF inhibitor is not 4-(2-amino-1H-imidazol-4-yl)-N-(tridecan-7-yl)butanamide or 4-(2-amino-1H-imidazol-4-yl)-N-tridecylbutanamide.

The persister bacterial cell or antibiotic resistant bacterial cell can be any bacteria described herein. In certain embodiments, the persister bacterial cell or the antibiotic resistant bacterial cell is a Gram-negative persister bacterial cell or Gram-negative antibiotic resistant bacterial cell. Exemplary persister bacterial cell or antibiotic resistant bacterial cells include, but are not limited to, E. coli, K. pneumoniae, A. baumannii, P. aeruginosa, S. aureus, and S. typhimurium.

The imidazole-based antifungal agent can be as described herein. In certain embodiments, the imidazole-based antifungal agent is econazole or a pharmaceutically acceptable salt thereof.

The method for re-sensitizing the persister bacterial cell or an antibiotic resistant bacterial cell to an antibacterial can further comprise the step of contacting the cell with an antibacterial.

The method for re-sensitizing the persister bacterial cell or an antibiotic resistant bacterial cell to an antibacterial can be conducted in vitro or in vivo.

### Genes in the Psp Family are Over-Expressed During Starvation

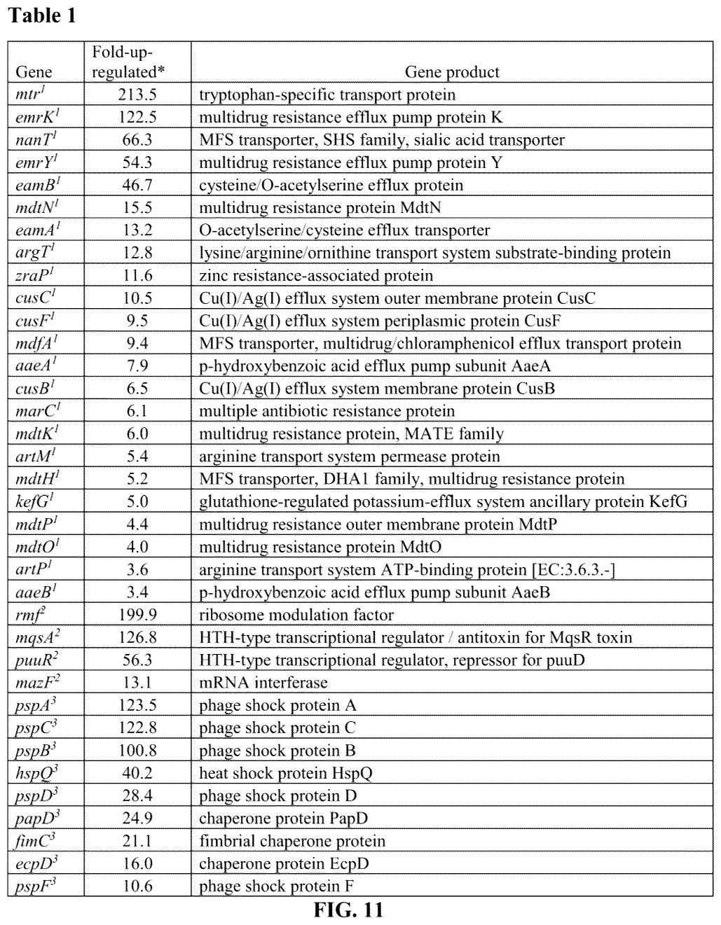

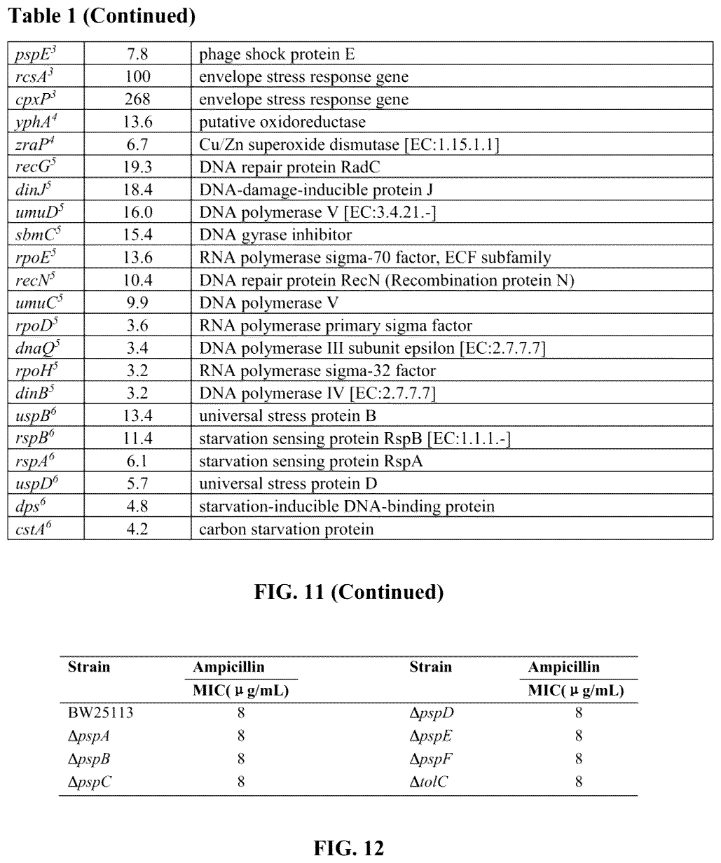

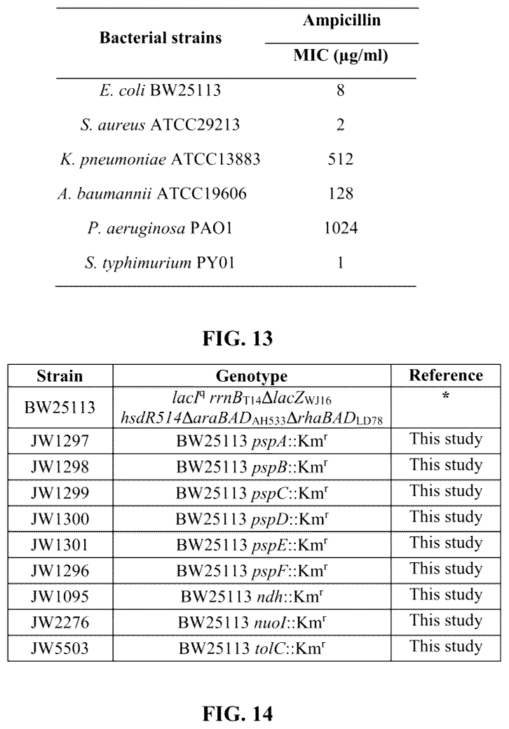

In order to explore the range of physiological responses that play an active role in development and maintenance of antibiotic tolerance during starvation, a RNA-Seq was first performed upon E. coli BW25113 to identify genes whose expression level was significantly up-regulated even after the test organisms had experienced a prolonged starvation episode. Since metabolic activities are reduced to a minimum when nutrients are depleted, the expression level of most functional genes is expected to be kept at a minimum, with the exception of essential proteins which may modulate adaptive physiological responses. Such proteins are therefore expected to contribute directly or indirectly to formation of starvation-induced tolerance. Based on the RNA-Seq data, a total of 58 genes were identified, which when compared to exponentially growing cells, were expressed at a level three-folds or more when the test organisms had encountered starvation stress for 24 hrs in physiological saline (FIG. 11). These genes included those encoding transcriptional regulators, membrane transporters, oxidative enzymes, DNA repair proteins and starvation stress sensors, among them, a functionally important gene cluster in which the expression level of all members was significantly elevated was identified. This gene cluster is the psp family which comprises the pspA, B, C, D, E and F genes, the expression level of which was up-regulated 124, 101, 123, 28, 8 and 11 folds respectively upon encountering starvation for 24 hrs (FIG. 11). Products of the psp operon are known to be able to sense a change in PMF, membrane-stored curvature elastic stress, presence of mis-localized secretins and other factors; activation of the psp operon enables the bacterial cells to maintain PMF or avoid mis-localized secretin-induced toxicity. The role of this gene cluster in mediating expression of bacterial tolerance response during nutrient starvation was then investigated.

### The Role of Psp Response in Mediating Starvation-Induced Tolerance

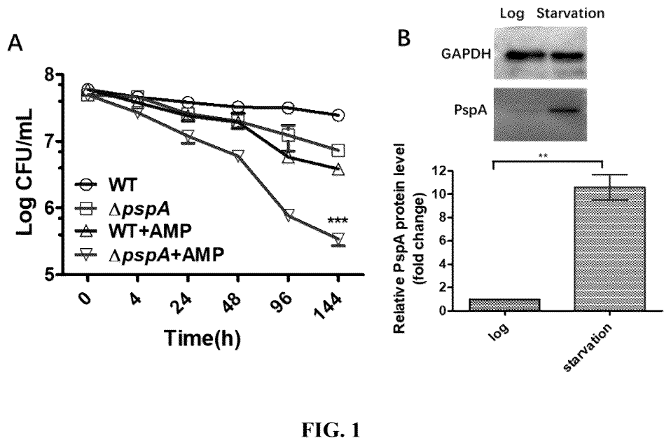

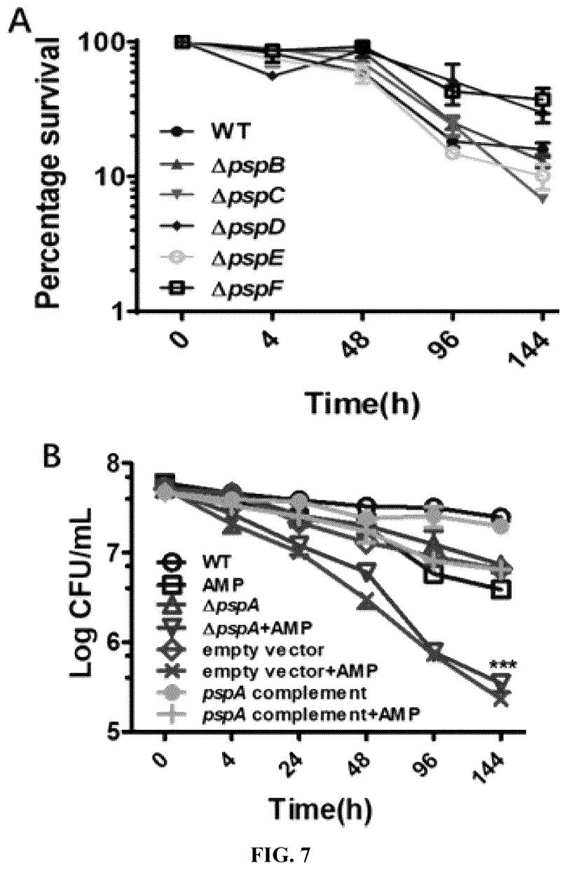

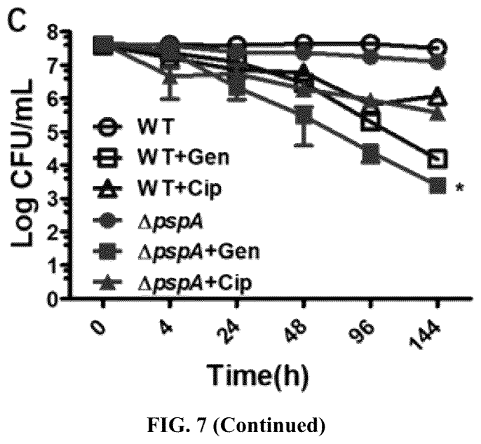

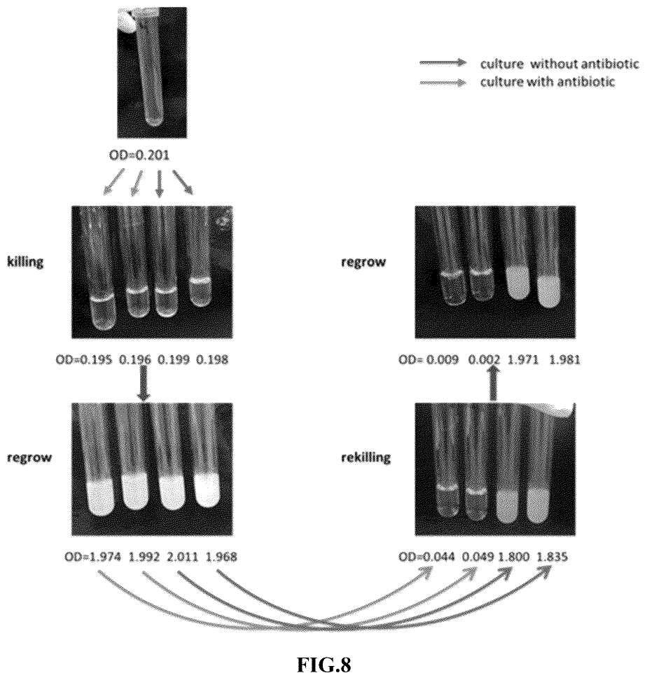

To test whether products of the up-regulated psp genes play a role in formation of antibiotic tolerance, the change in level of starvation-induced tolerance of specific gene knockout mutants to the wild type strain within a six days period were monitored and compared. It was noticed that, although the level of tolerance in both the wild type and ΔpspA strain was similar at the initial phase of treatment, the proportion of ampicillin tolerant cells in the ΔpspA mutant dropped at a significantly faster rate over the six days period when compared to the wild type. The size of the tolerant sub-population in knockout mutants of the other genes in the psp family, however, was similar to that of wild type throughout the experiment (FIG. 1A; FIG. 7A). The size of the antibiotic tolerant sub-population in the ΔpspA strain dropped to ˜3×10⁵ cells/mL after 6 days of ampicillin treatment, which was only 10% of that of wild type (4×10⁶ cells/mL). These findings imply that the pspA gene product is not essential for tolerance formation but required for long term maintenance of the tolerance phenotype. The size of survival population after ampicillin treatment remained the same as that of the wild type strain when a plasmid-borne pspA gene was introduced into the pspA gene deletion strain in gene complementation experiment (FIG. 7B). It was also confirmed that the phenotypes of the tolerant sub-population were not due to genetic mutations that conferred drug resistance upon removal of antibiotic stress, as the tolerant sub-population was able to regrow as antibiotic susceptible organisms, and the minimum inhibitory concentration (MIC) of knockout mutants of the psp gene family remained the same as that of wild type (8 μg/ml) (FIG. 8; FIG. 12). PspA is the key functional protein among members of the Psp family and known to play a major role in Psp response. Its role in maintenance of the tolerance phenotype was next investigated and tested whether an increase in gene expression of pspA actually resulted in a corresponding increase in protein level. Western blotting was performed, with results showing that PspA was barely detectable in the exponential phase, yet an abundance of this protein was synthesized upon encountering starvation for 24 hrs, the level of which was about 9.4 folds that of the exponential phase control (FIG. 1B). In addition to maintenance of antibiotic tolerance, the pspA gene product was also found to have effect on bacterial survival during starvation in the absence of antibiotics. Throughout a period of starvation for six days without ampicillin treatment, the population size of the pspA knockout strain shrank gradually to a level of ˜7×10⁶ cells/mL, whereas that of wild type remained relatively constant at ˜2.5×10⁷ cells/mL (FIG. 1A). Apart from ampicillin, the sizes of tolerant sub-population against gentamicin and ciprofloxacin were also compared between ΔpspA and wild type. The size of the ΔpspA population that survived after gentamicin treatment was found to decrease, but remained at a high level after treatment with ciprofloxacin (FIG. 7C).

### PspA Protein Plays a Role in Maintaining PMF During Starvation

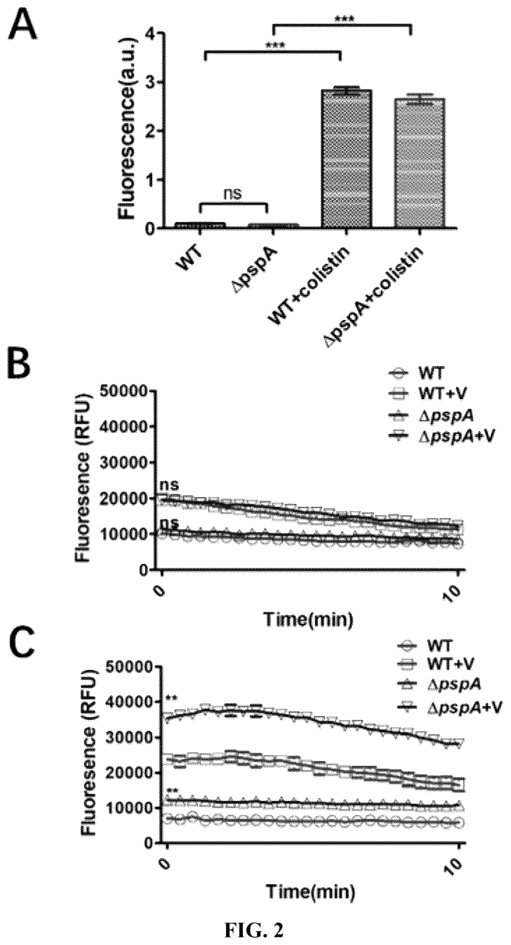

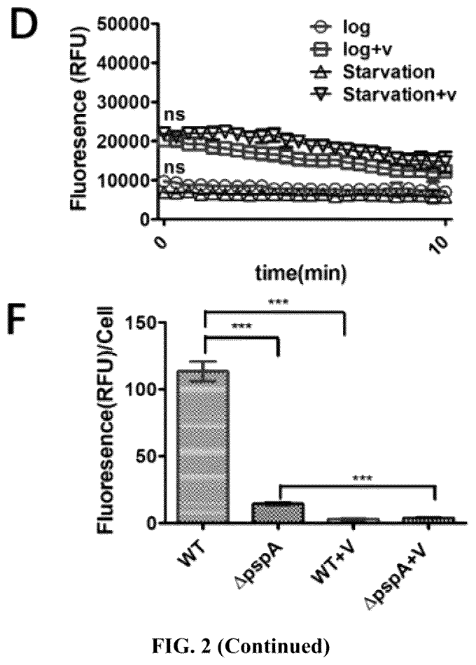

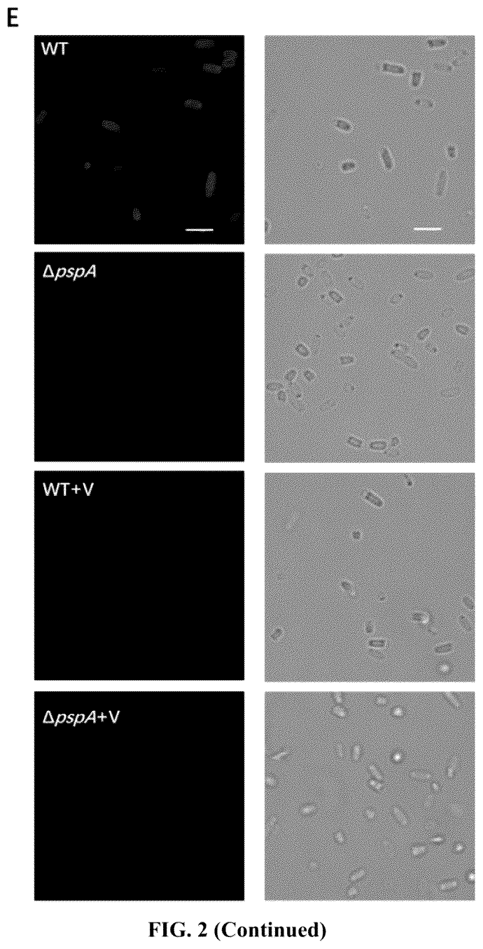

It was reported that Psp proteins were involved in a wide range of membrane functions, with the PspBC complex being located in the inner membrane, interacting with PspA to prevent alteration in inner membrane permeability and cytoplasmic shrinkage. It was therefore hypothesized that deleting the pspA gene may undermine membrane integrity, leading to membrane leakage. By using the dye SYTOX Green to test membrane permeability during starvation, however, it was showed that the amount of dye taken up by the wild type and ΔpspA strain during starvation was similar (FIG. 2A), indicating that membrane permeability was not significantly altered in the ΔpspA mutant. Likewise, although colistin treatment was found to cause membrane damage and an eventual increase in membrane permeability, the degree of changes in membrane permeability in both wild type E. coli and ΔpspA mutant after colistin treatment were similar (FIG. 2A), suggesting that the Psp response conferred little protective effect against this membrane destabilizing agent.

One major role of the PspA protein is to maintain bacterial PMF. Oligomers of PspA other than the PspBCA complex were found to bind to membrane phospholipids and prevent proton leakage. It was then postulated that the reason why increased PspA expression could help maintain phenotypic tolerance is that it helped preserve PMF during starvation. The dye DiSC₃(5) was used to test the extent of changes in bacterial cell membrane potential upon entry into the starvation mode. High level accumulation of the dye in the bacteria cells would result in quenching of the overall fluorescence of the cell culture, whereas rapid release of the dye into the medium would result in dequenching upon depolarization of the dye. In the exponential phase, the fluorescence intensities recorded for the wild type and ΔpspA strains were found to be similar with valinomycin as positive control since it caused dissipation of membrane potential and then a sharp increase in fluorescence (FIG. 2B). Upon encountering starvation for 24 hrs, however, the fluorescence intensity of the ΔpspA strain was significantly higher than that of the wild type, indicating that the amount of dye accumulated intracellularly was much lower in the pspA mutant during starvation (FIG. 2C). On the other hand, the fluorescence intensity of the wild type strain also remained at a similar level between exponential phase and 24 hrs starvation, thereby confirming that PMF of the wild type could be maintained at a level equivalent to that of the exponential phase during starvation (FIG. 2D). These findings were consistent with results of the confocal microscopy experiment, in which only the wild type strain could be stained by DiSC₃(5) upon starvation for 24 hrs. The dye apparently could not enter cells of the ΔpspA strain because the membrane potential was too low (FIG. 2E). Based on the confocal images, it was calculated that the fluorescence intensity of each group and found that the intensity of the wild type strain (100 RFU/cell) was approximately seven folds that of the ΔpspA mutant strain (15 RFU/cell); such finding further confirmed that knocking out the pspA gene would cause rapid dissipation of PMF during nutrient starvation (FIG. 2F). Our data indicated that PMF was maintained under starvation condition as PspA was highly expressed and preserved PMF. This observation is consistent with the previous finding that PMF was advantageous for the survival of hypoxic, non-growing bacteria or those under nutrient starvation conditions.

### Maintenance of PMF is Essential for Long Term Survival of Starvation-Induced Tolerant Cells

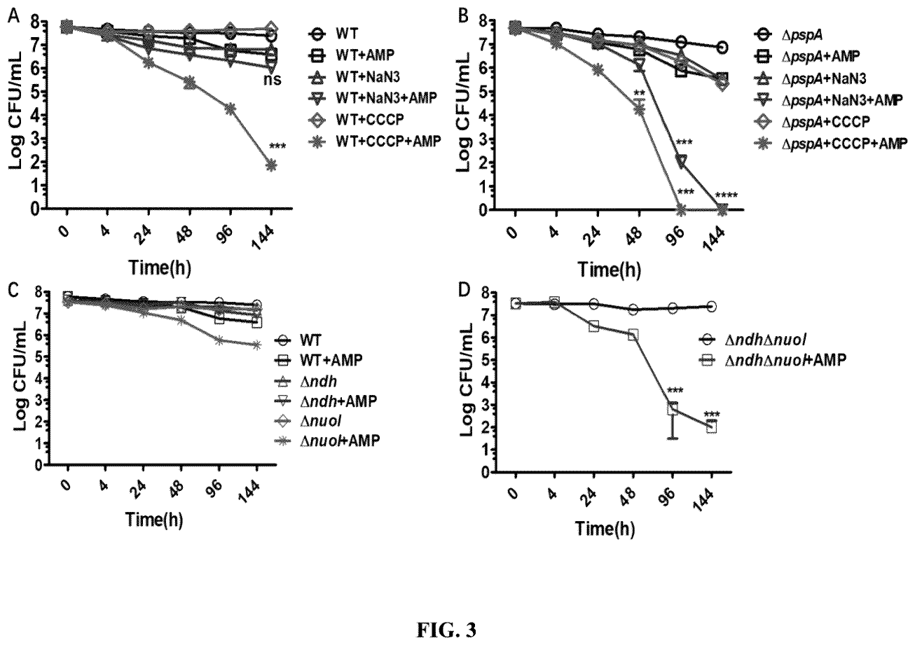

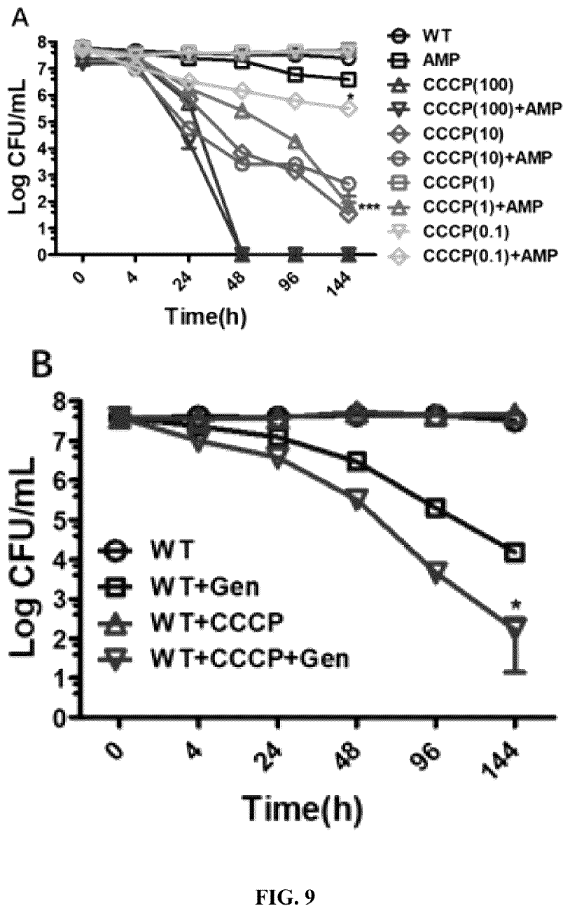

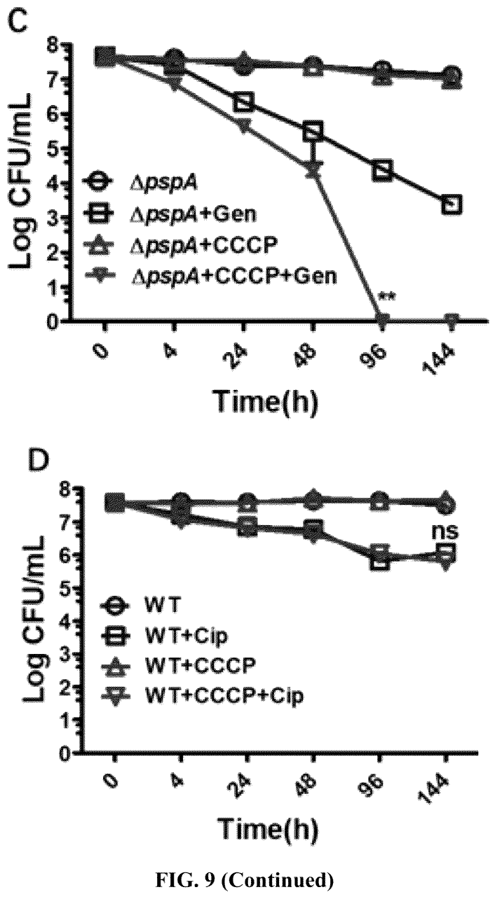

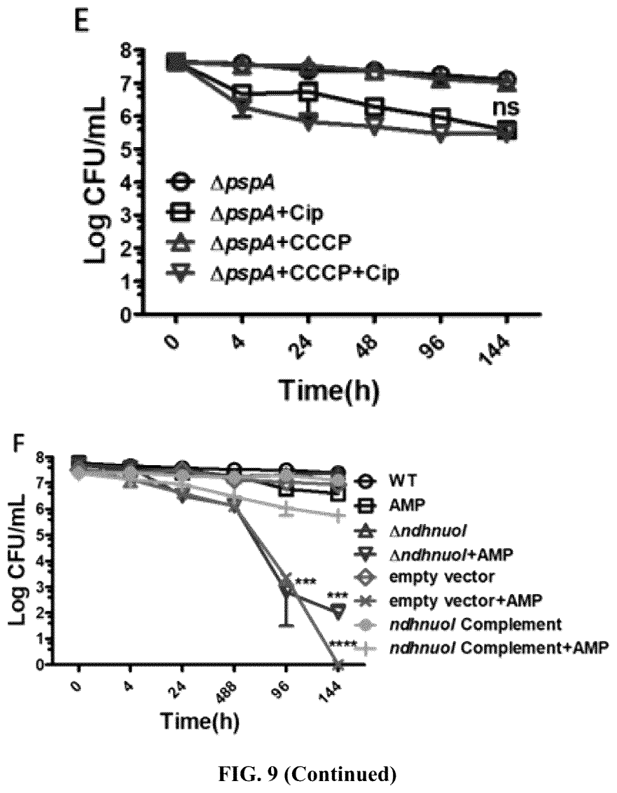

Upon identifying the PMF maintenance role of the pspA gene product and confirming the functional importance of PMF in actively maintaining phenotypic tolerance, it was hypothesized that merely preventing dissipation of pre-existing PMF was not sufficient for totally abolishing the ability to maintain the tolerance phenotype as bacteria subjected to starvation stress still undergo a low level of oxidative phosphorylation to generate a basal level of PMF. To test this possibility, it was determined whether sodium azide, which inhibits cytochrome C oxidase and hence the ability to generate PMF, could cause significant reduction in the tolerance level of bacterial population subjected to prolonged starvation. The results showed that the population size of the wild type strain was only slightly reduced upon treatment with sodium azide, regardless of whether ampicillin was present or not (FIG. 3A). The effect of sodium azide alone on the ΔpspA mutant was similar to that of the wild type strain, exhibiting a slight bactericidal effect. In the presence of ampicillin, however, sodium azide was able to eradicate the entire tolerant sub-population at 144 hrs (FIG. 3B). These findings therefore confirm that, under nutrient-deficient conditions, PMF is essential for prolonged maintenance of the tolerance phenotype, as simultaneous inhibition of both PMF production and maintenance, by sodium azide treatment and deletion of the pspA gene respectively, led to complete eradication of ampicillin tolerant cells that formed under prolonged starvation conditions. To further confirm the role of PMF in starvation-induced tolerance response, the uncoupling agent carbonyl cyanide-m-chlorophenylhydrazone (CCCP), which is a known protonophore, was used to test its effect on E. coli tolerance response. When CCCP (1 μM) was added to E. coli cells which had been starved for 24 hrs, the size of the surviving population remained unchanged throughout the 144 hrs experiment, but the population size dropped to ˜80 cells/mL when ampicillin was present (FIG. 3A). When the concentration of CCCP was 10 μM, the size of the E. coli population recorded after CCCP treatment for 144 hrs dropped to the range of ˜50 cells/mL and ˜300 cells/mL regardless of whether ampicillin was present (FIG. 9A). If the concentration of CCCP was increased to 100 μM, the size of the E. coli population which had been starved for 24 hrs dropped from ˜2.5×10⁷ cells/mL to ˜5×10⁵ cells/mL within 24 hrs, and to ˜2×10⁴ cells/mL if ampicillin was also present. The entire antibiotic tolerant population was then eradicated by 48 hrs with or without ampicillin treatment, indicating that starvation-induced tolerant cells could no longer survive for a prolonged period upon collapse of PMF (FIG. 9A). Likewise, phenotypic tolerance to gentamicin cannot be maintained upon PMF dissipation (FIG. 9B, C). However, tolerance to ciprofloxacin was not affected by CCCP treatment (FIG. 9D, E). Taken together, the data showed that active maintenance of PMF is the key mechanism underlying prolonged expression of phenotypic antibiotic tolerance during nutrient starvation.

To confirm if active maintenance of PMF indeed plays a key role in expressing phenotypic tolerance in bacteria, it was further tested if disruption of the cellular mechanisms governing PMF formation could affect tolerance formation. ETC plays an important role in generating PMF. Two enzymes, namely NADH dehydrogenase I and NADH dehydrogenase II, which are encoded by the genes nuoI and ndh respectively, are key components of the ETC. Upon starvation for 24 hrs and then six days of ampicillin treatment, the population size of the E. coli strains BW25113::ΔnuoI and Δndh was found to drop to ˜3.5×10⁵ cell/mL and ˜-8×10⁶ cell/mL respectively (FIG. 3C). Importantly, the size of the tolerant population of the E. coli BW25113::ΔndhΔnuoI strain, in which both genes were simultaneously deleted, dropped sharply to ˜200 cells/mL, a ˜10⁴ fold reduction, upon treatment with ampicillin for six days, suggesting that inhibition of activities of ETC components indeed severely affects production and maintenance of bacterial PMF, and hence the long term survival of tolerant cells that are exposed to ampicillin (FIG. 3D; FIG. 9F).

Active Efflux Driven by PMF Contributes Partially to Maintenance of Starvation-Induced Tolerant Cells in Escherichia coli

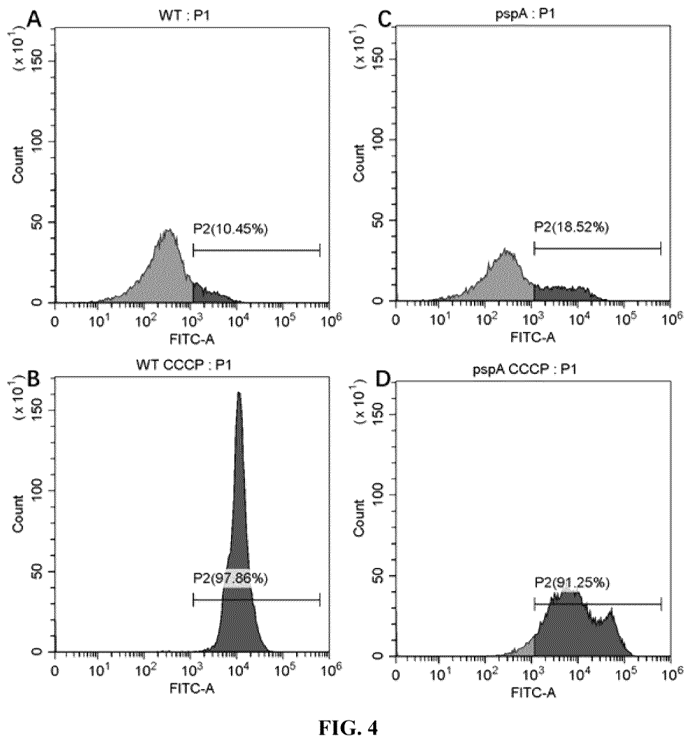

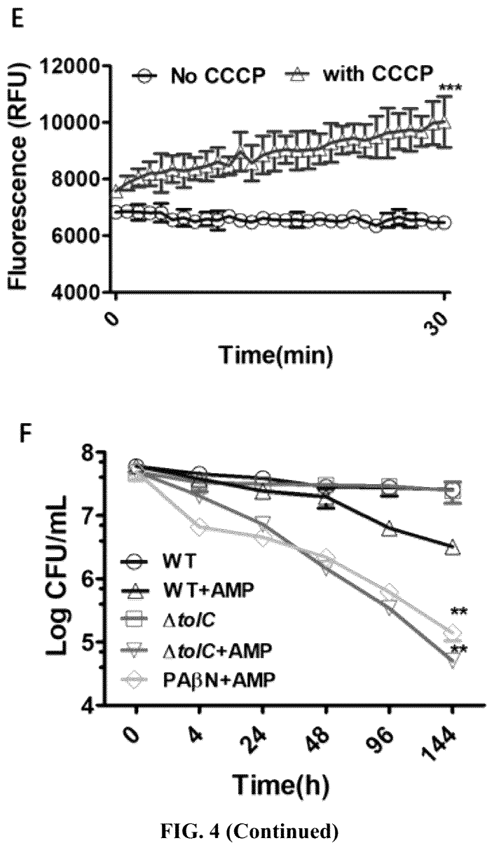

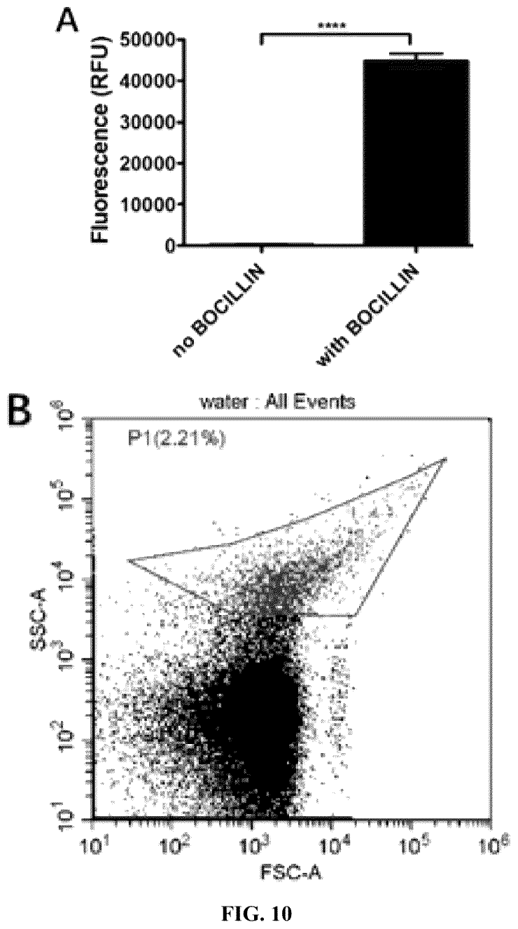

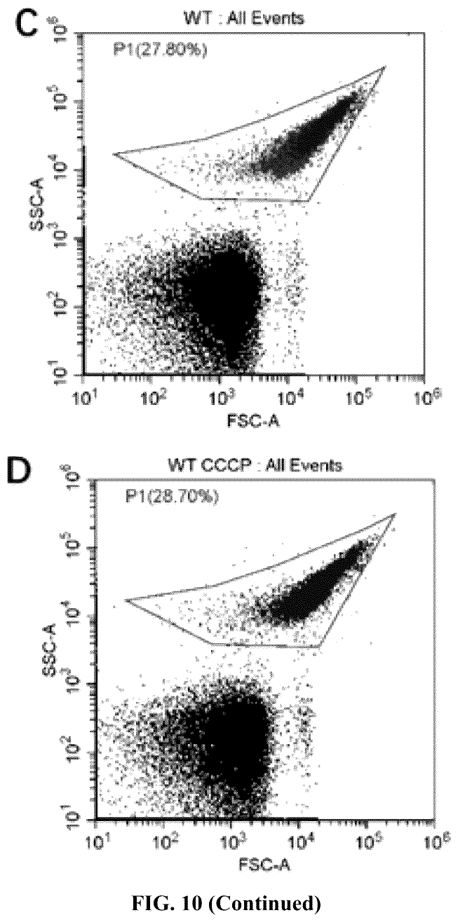

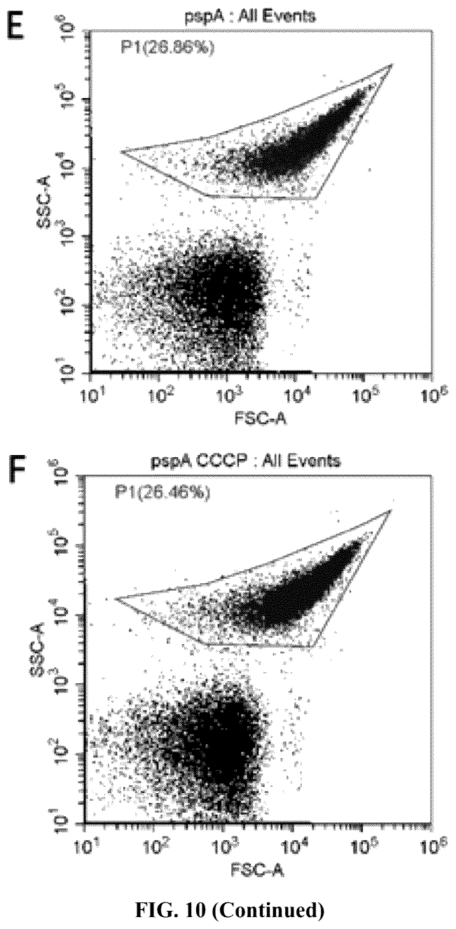

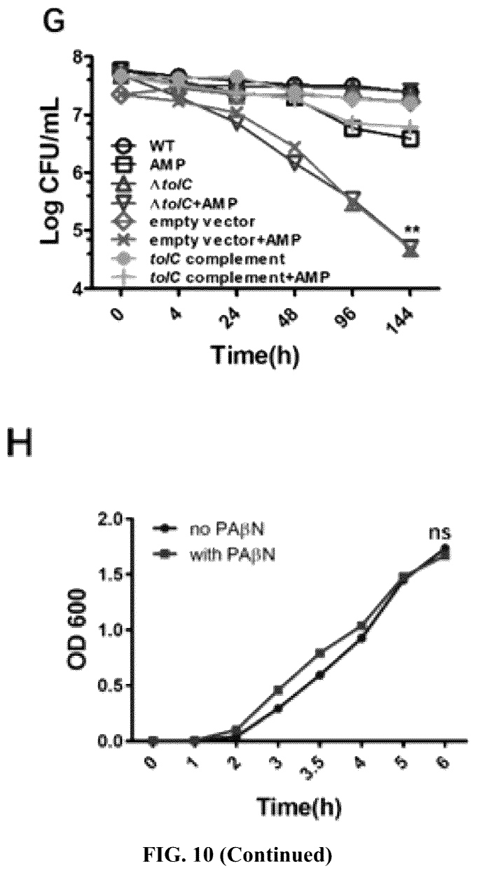

PMF is involved in numerous cellular functions; in particular, it plays an essential role in maintaining efflux activities. Bacterial efflux could lead to decrease in antibiotic accumulation, thereby facilitating the cells to form tolerant cells and survive from antibiotic treatment. The role of PMF in maintaining the antibiotic tolerance phenotype was due to its effect on promoting efflux activities was tested. A fluorescent β-lactam antibiotic known as BOCILLIN™ FL Penicillin (BOCILLIN) was used to depict the degree of accumulation of β-lactam antibiotic in the presence and absence of CCCP. It was first confirmed that CCCP had little effect on the overall fluorescence signal as the fluorescence level exhibited by CCCP itself was only ˜250 RFU, or ˜180 times less than that of BOCILLIN (˜45000 RFU) (FIG. 10A). In this experiment, flow cytometry was performed to assess the degree of accumulation of BOCILLIN with or without CCCP treatment. Wild type bacterial cells which have been subjected to starvation for 24 hrs, followed by CCCP treatment, were generally well-stained by BOCILLIN with 97.86% of the cells exhibiting high BOCILLIN intensity (>10³ RFU), whereas the fluorescence level recorded in the absence of CCCP was significantly lower, with only 10.45% of the cells exhibiting high BOCILLIN intensity (>10³ RFU), indicating that the amount of β-lactam antibiotic accumulated intracellularly increased upon PMF dissipation (FIG. 4A, B; FIG. 10B, C, D). The percentage of ΔpspA cells that exhibited high BOCILLIN level was 18.52%, which was more than that of the wild type (10.45%) (FIG. 4 C, D; FIG. 10E, F). This finding shows that the level of intracellular β-lactam antibiotic accumulation increases if PMF cannot be properly maintained. It was then determined whether accumulation of BOCILLIN associated with artificial dissipation of PMF during starvation was due to failure to undergo efflux. The dye Nile Red, a common substrate of efflux pumps, was used in investigation of bacterial efflux activities. In this experiment, Nile Red was incubated with the 24 hrs-starvation population for 30 mins, followed by addition of CCCP and fluorescence measurement. The results showed that collapse of PMF upon addition of CCCP correlated well with an increase in fluorescence signal. Specifically, the fluorescence intensity in the wild type strain increased from ˜6500 RFU to ˜10000 RFU within 30 mins, indicating a diminished efflux efficiency in the absence of PMF (FIG. 4E).

Tolerance formation was previously shown to negatively correlate with intracellular β-lactam accumulation. To further determine if efflux activities were indeed involved in starvation-induced antibiotic tolerance, deleting the tolC gene, the product of which constitutes a key component of several major efflux systems, such as AcrAB-TolC and EmrAB-TolC, resulted in reduction in the size of antibiotic-tolerant population recorded during starvation was tested. Under the assay condition, the size of the tolerant population in the E. coli ΔtolC mutant (˜5×10⁴ cells/mL) was much smaller than that of wild type (˜2.5×10⁷ cells/mL) upon treatment with ampicillin for six days, suggesting that efflux pumps played a role in expression of the antibiotic tolerance phenotype (FIG. 4F; FIG. 10G). This idea was further confirmed by testing the effect of PAβN, an efflux pump inhibitor, which was also found to cause a significant drop in the size of tolerant population (˜1.5×10⁵ cells/mL) (FIG. 4F). It was shown that PAβN did not exert any negative effect on bacterial growth, indicating that the tolerance suppression effects conferred by this compound was not due to its bactericidal effect (FIG. 10H). These data suggested that efflux pump activity, which might be maintained through the PMF, contributed to expression of phenotypic antibiotic tolerance during nutrient starvation.

Through comparison between the effect of PMF dissipation and efflux suppression on the survival of starvation-induced tolerant cells, it was found that disruption of PMF exhibited a much stronger effect on tolerance suppression than inhibiting efflux activity. The entire tolerant cell population in the wild type strain was recorded as ˜50 cells/mL after six days of treatment with CCCP and ampicillin; in the case of pspA knockout, treatment with sodium azide/CCCP and ampicillin could cause complete eradication by 144 hrs. On the other hand, the size of the tolerant population in the wild type strain remained at ˜5×10⁴ cells/ml upon deletion of tolC or treatment with efflux pump inhibitor PAβN (FIG. 4F). These data strongly suggest that maintenance of PMF are key mechanisms underlying maintenance of starvation-induced bacterial antibiotic tolerance, and that the functional role of PMF probably lies in regulation of efflux and other important membrane transportation activities, which warrant further investigation.

### Maintenance of PMF is Key Tolerance Mechanism in Both Gram Negative and Positive Bacteria

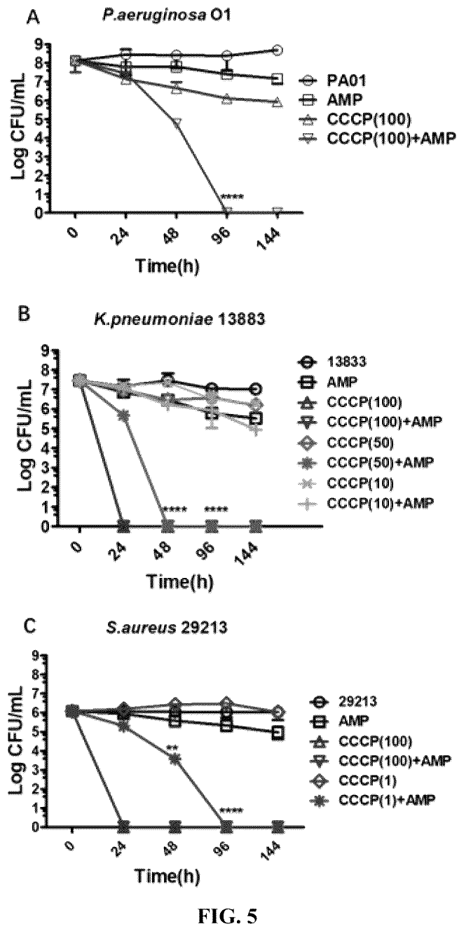

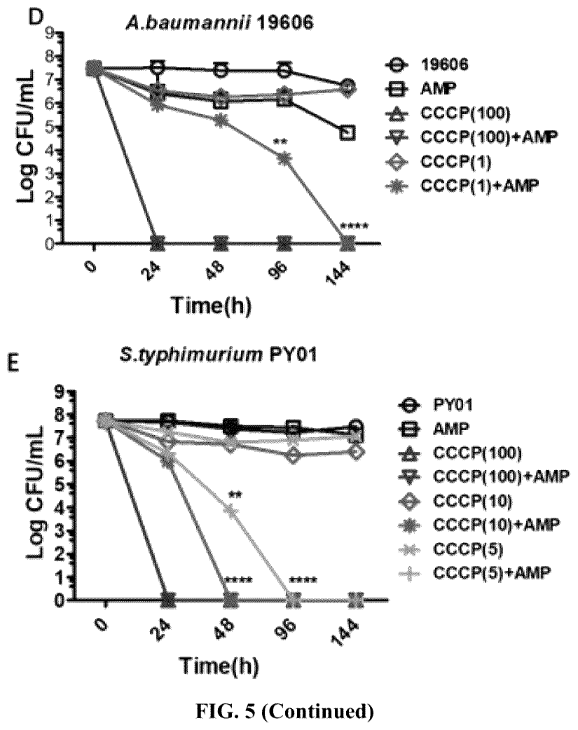

To determine if maintenance of PMF is an active cellular mechanism universally employed by various bacterial species to promote tolerance formation, it was tested whether CCCP could eradicate starvation-induced tolerant cells of major bacterial pathogens. The data confirmed that, a low concentration of CCCP was sufficient to suppress or even completely abolish phenotypic ampicillin tolerance in K. pneumoniae, S. aureus, A. baumannii and S. typhimurium, and that higher concentration of CCCP (100 μM) could eradicate tolerant bacteria cells even in the absence of ampicillin (FIG. 5B-E). The tolerant sub-population were eradicated by ampicillin in the presence of 100 μM CCCP in P. aeruginosa (FIG. 5A). This finding confirmed that PMF maintenance is a universal mechanism underlying prolonged expression of phenotypic antibiotic tolerance in most bacterial species.

### Econazole Acts as Antibiotic Adjuvant to Kill Starvation-Induced Bacterial Tolerant Cells

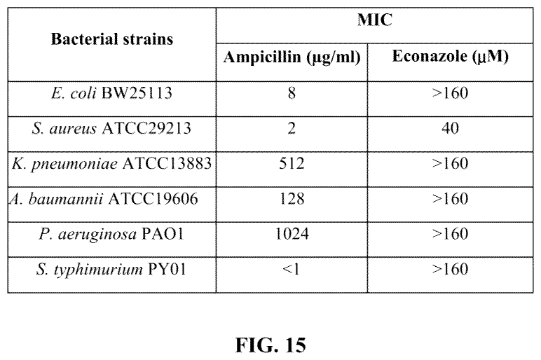

An FDA approved drug library was screened by performing a tolerance assay to select compounds that act synergistically with ampicillin to kill E. coli tolerant cells generated by incubating log phase E. coli cell in saline for 24 hours. An antifungal drug, econazole, was identified that effectively kills starvation-induced E. coli tolerant cells in the presence of ampicillin. Compared with an initial population size of ˜4×10⁷ CFU/mL recorded upon suspension and incubation with saline for 96 hrs, the population size remained at a high level of ˜-1×10⁶ CFU/mL after treatment with a lethal dose ampicillin (10×MIC) for 96 hrs, indicating that the vast majority of the bacterial population were tolerant to ampicillin. However, the entire tolerant population was eradicated upon treatment with a combination of econazole and ampicillin for 24 hrs (FIG. 15, FIG. 16A). This killing effect could be attributed to the synergistic actions of econazole and ampicillin, as econazole alone had little effect on tolerant cells, even though the population size decreased slightly at the early phase of econazole treatment (˜1×10⁴ CFU/mL at 24 hrs). Further characterization of the effect of the econazole and ampicillin combination on different bacterial species showed that at 40 μM, econazole alone only exhibited slight killing effect on tolerant cells of Gram-negative bacteria such as K. pneumoniae, A. baumannii P. aeruginosa and S. typhimurium, but eradicated tolerant cells of Gram-positive bacteria (S. aureus) within 96 hrs (FIG. 16B-16F). This discrepancy may be due to the difference between the cell wall structure of Gram positive and Gram negative bacteria, and is consistent with the finding that the MIC of econazole in the S. aureus (40 μM) is much lower than in Gram negative strains (>160 μM) (FIG. 15). When used in combination with ampicillin, however, econazole exhibited significant killing effect on tolerant cells of K. pneumoniae and A. baumannii, eradicating the entire tolerant sub-population within 24 hrs (FIG. 16B, 16C). For P. aeruginosa and S. typhimurium, combined usage of econazole and ampicillin could eradicate the tolerant sub-population by 96 hrs (FIG. 16D,16F). Econazole also exhibited synergistic antimicrobial effect when used in combination with other β-lactam drugs; for example, the size of the tolerant population was reduced to ˜500 CFU/mL within 96 hrs in the presence of econazole and ceftazidime, whereas that recorded at 96 hrs upon treatment with ceftazidime alone remained at ˜1.4×10⁷ CFU/mL (FIG. 16F).

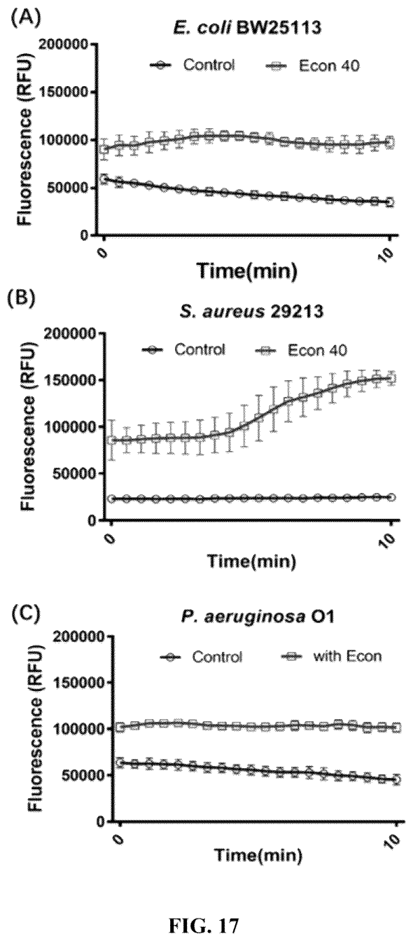

The effect of econazole on the cytoplasmic membrane of bacterial cells was investigated by measuring the transmembrane electric potential with the use of the fluorescent probe DiSC₃(5). This dye accumulates in bacterial cells and results in self-quenching of the overall fluorescence of the cell suspension. Upon depolarization, the dye is rapidly released into the medium, resulting in dequenching that can be detected fluorometrically. The effect of econazole in E. coli, S. aureus and P. aeruginosa was tested. The fluorescence signal was found to increase after adding econazole into the bacteria cells, indicating that econazole caused dissipation of PMF in both Gram negative and Gram positive strains (FIG. 17). It was hypothesized that dissipation of membrane potential of bacterial tolerant cells would lead to cell death. CCCP (carbonyl cyanide m-chlorophenylhydrazone) is a known PMF dissipator and was used to investigate whether it could kill bacterial tolerant cells in a manner similar to that of econazole. Out data showed that CCCP alone and a combination of CCCP and ampicillin could both kill bacterial tolerant cells of E. coli, however the killing effect of a combination of CCCP and ampicillin was significantly stronger than that of CCCP alone (FIG. 16G). This finding confirmed that dissipation of bacterial PMF could sensitize bacterial tolerant cells to antibiotics. It should be noted that CCCP and econazole by itself does not exhibit antibacterial activity on major Gram negative bacterial species, with MIC of >160 μM being recorded in all test strains. For the Gram positive pathogen S. aureus, a MIC of 40 μM was recorded (FIG. 15). CCCP is toxic to human, yet econazole is FDA-approved and has been proven safe for use as a therapeutic agent, including systematic administration in human, suggesting this compound has high potential to be developed into clinical therapy to kill bacterial tolerant cells.

It was then tested if tolerant cells could be eradicated by treatment with econazole alone or in combination with various types of commonly used antibiotics. In E. coli, the size of bacterial population that survived was about 200 CFU/mL upon treatment with a combination of econazole and ciprofloxacin/gentamicin for 4 hrs, whereas the size of the population that survived treatment with econazole alone was about 1.7×10⁵ CFU/mL, which was similar to that recorded without treatment (population resuspended in saline, ˜6.3×10⁷ CFU/mL). When the treatment time reached 24 hrs, all tolerant cells were killed by a combination of econazole and ciprofloxacin/gentamicin (FIG. 16H). These findings indicate that, apart from ampicillin, econazole also enhanced the killing effect of quinolones and aminoglycosides against bacterial antibiotic tolerant cells. It was also found that econazole could act in combination with a sub-lethal dose of meropenem to eradicate the tolerant sub-population of carbapenem resistant E. coli J53 strain which expressed the carbapenemase NDM-1, when such cells were subjected to nutrient starvation (FIG. 16I). This finding indicates that econazole can enhance the killing effect of conventional antibiotics on the tolerant sub-population of not only antibiotic susceptible strains, but also that of resistant organisms.

### Morphology of Econazole-Treated Tolerant Cells

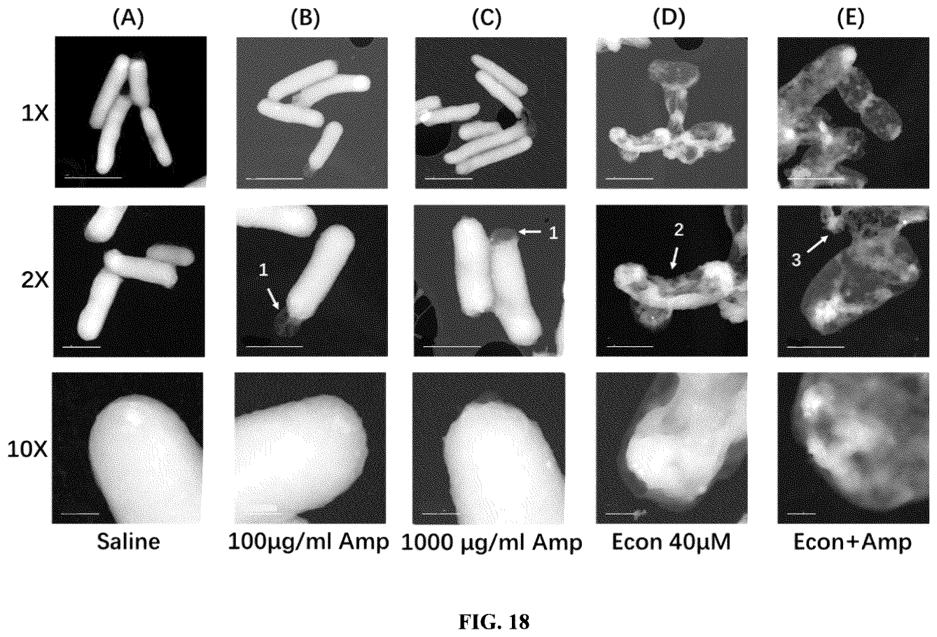

The effects of the econazole and ampicillin combination on the cellular structure of E. coli tolerant cells were further investigated by scanning electron microscopy (SEM). Upon treatment with a high dose of ampicillin (100 μg/ml and 1000 μg/ml), tolerant cells exhibited slight shrinkage in the pole areas, but the microscopy image of the membrane remained as sharp and smooth as cells treated with saline (FIG. 18A, 18B, 18C). However, tolerant cells treated with 40 μM econazole exhibited a rough cell surface, as well as leakage of intracellular material characterized by an increasingly transparent cytosol (FIG. 18D). When treated with a combination of econazole and ampicillin, the cell membrane structure was severely damaged, leading to complete leakage of intracellular contents and cell lysis, with a feature of complete transparency of the cells visible under SEM (FIG. 18E). It should also be noted that econazole was not found to cause any detectable membrane damage or morphological changes in exponentially growing cells which actively undergo aerobic respiration to generate a strong PMF (data not shown), suggesting that its deleterious effects on starvation-induced antibiotic tolerant cells were due to PMF dissipation. These findings are consistent with the prolonged killing data in that bacteria under starvation were initially tolerant to ampicillin, but could be completely eradicated by the econazole and ampicillin combination (FIG. 16).

### Econazole and Ceftazidime Combination Eradicates Tolerant Bacterial Population In Vivo

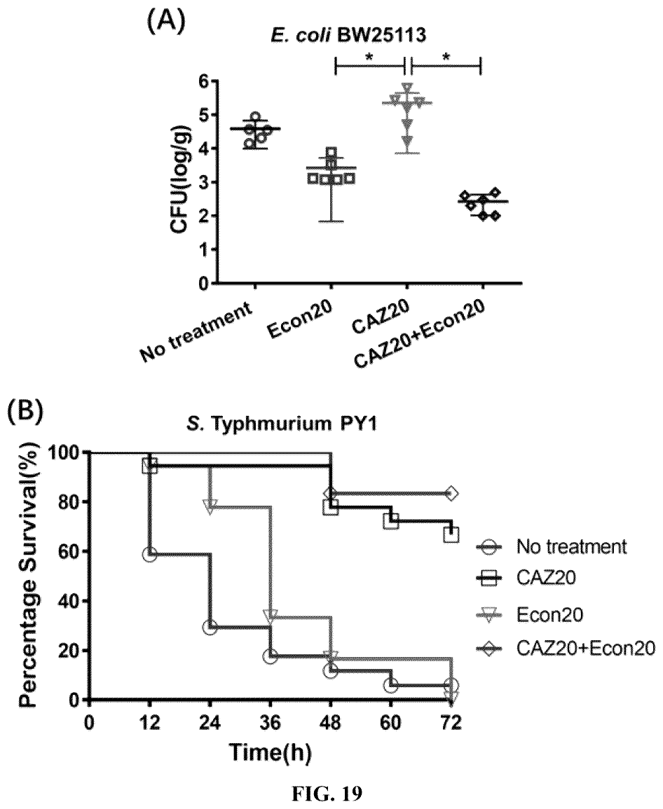

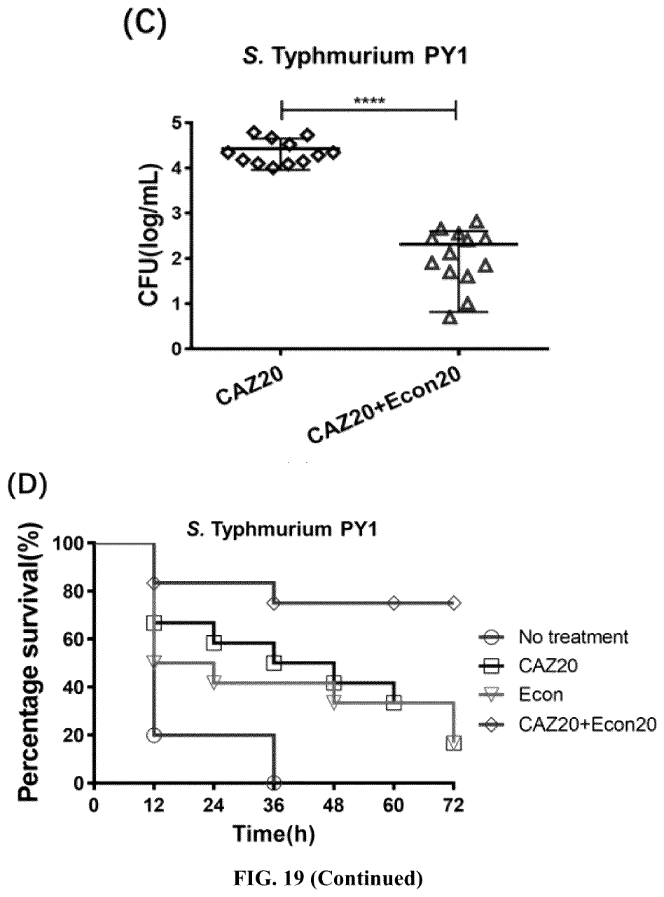

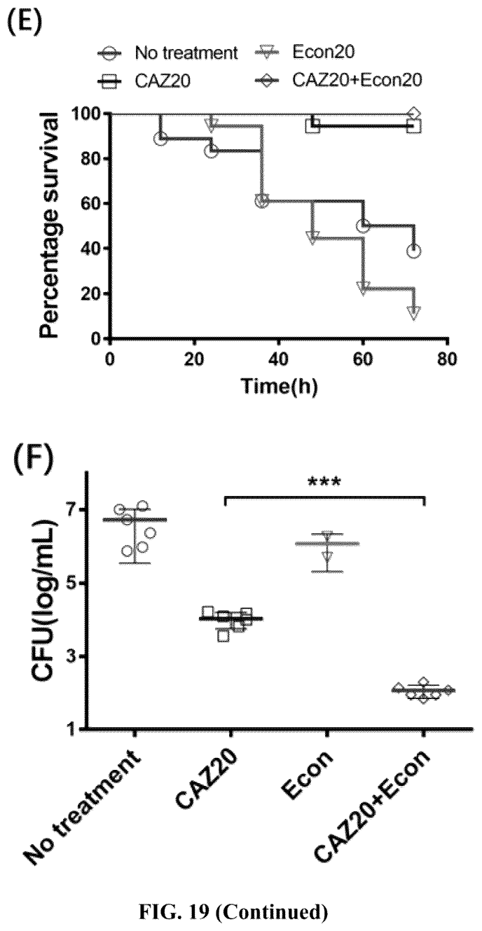

The efficacy of the β-lactam and econazole combination in eradicating bacterial tolerant cells was further tested in a mouse infection model, with ceftazidime, an antibiotic commonly used in clinical treatment of bacterial infection, being the test agent. First, a deep-seated thigh tolerance model using E. coli BW25113 as the test organism was established; the data showed that treatment with econazole alone (20 mg/kg), or a combination of econazole (20 mg/kg) and ceftazidime (20 mg/Kg), resulted in significantly more (P=0.026 and P=0.031 respectively) efficient eradication of E. coli tolerant cells than treatment with ceftazidime (20 mg/Kg) alone (FIG. 19A). Second, a peritonitis infection model was tested involving S. typhimurium PY1 and found that both ceftazidime and a combination of ceftazidime and econazole could protect the test animals from being killed by tolerant infection elicited by inoculation of 7.6×10⁵ CFU of S. typhimurium PY1, with the combination therapy being slightly more effective (FIG. 19B). After treatment for 72 h, CFU of S. typhimurium PY1 in the surviving animals were determined, with results showing that the combination therapy caused significantly higher (P<0.0001) rate of eradication of PY1 tolerant cells in mice (FIG. 19C). Similar effects were seen in mice infected with a lower dose (2.8×10⁵ CFU) of S. typhimurium PY1 (FIG. 19E, F). When infected with a relatively high dose of S. typhimurium PY1 (1.5×10⁶ CFU), however, 80% survival rate was recorded among mice treated with a combination of econazole (20 mg/kg) and ceftazidime (20 mg/Kg), whereas treatment with ceftazidime alone (20 mg/kg) could only rescue 10% of the infected mice (FIG. 4D). These findings confirm that econazole could significantly enhance the efficacy of ceftazidime in eradicating Salmonella tolerant cells in vivo.

Antibiotic tolerance is the phenomenon in which a sub-population of bacteria survive against lethal dosages of antibiotic treatment and re-grow upon withdrawal of the drug. In this work, one aim was to delineate active tolerance mechanisms in bacteria. Through systematic analysis of the gene expression profile of bacteria subjected to prolonged starvation, it showed that products of the psp gene family played a role in preventing dissipation of PMF, thereby facilitating proper functioning of specific efflux and transportation systems even during nutrient starvation. It was demonstrated that such cellular activities are essential for maintaining the survival fitness of the antibiotic tolerant sub-population. Discovered by Peter Model in 1990, the PspA protein was first shown to be induced in Escherichia coli upon infection by the filamentous phage f1. Psp proteins have since been postulated to play a role in regulating bacterial virulence, maintenance of PMF and mediation of envelope stress response. The resA and cpxP genes, which mediate bacterial envelope stress response and were also reported to play a role in maintaining PMF, were found to be up-regulated about 100 and 268 fold respectively in this work.

The Psp response was found to be involved in regulation of indole-induced tolerance, as the indole-induced tolerance sub-population size was reduced dramatically in the pspBC mutant. It has also been shown that PspA was over-expressed in stationary phase bacterial population, and that under alkaline conditions (pH 9), organisms lacking the pspABC genes exhibited significantly lower survival rate than wild type, suggesting that the Psp response can enhance bacterial survival under hostile conditions. Despite these findings, however, the functional importance of the Psp response in mediating expression of phenotypic antibiotic tolerance in bacteria appears to be overlooked. This work describes the essential role of PspA in mediating expression of starvation-induced antibiotic tolerance response through maintaining PMF in bacteria.

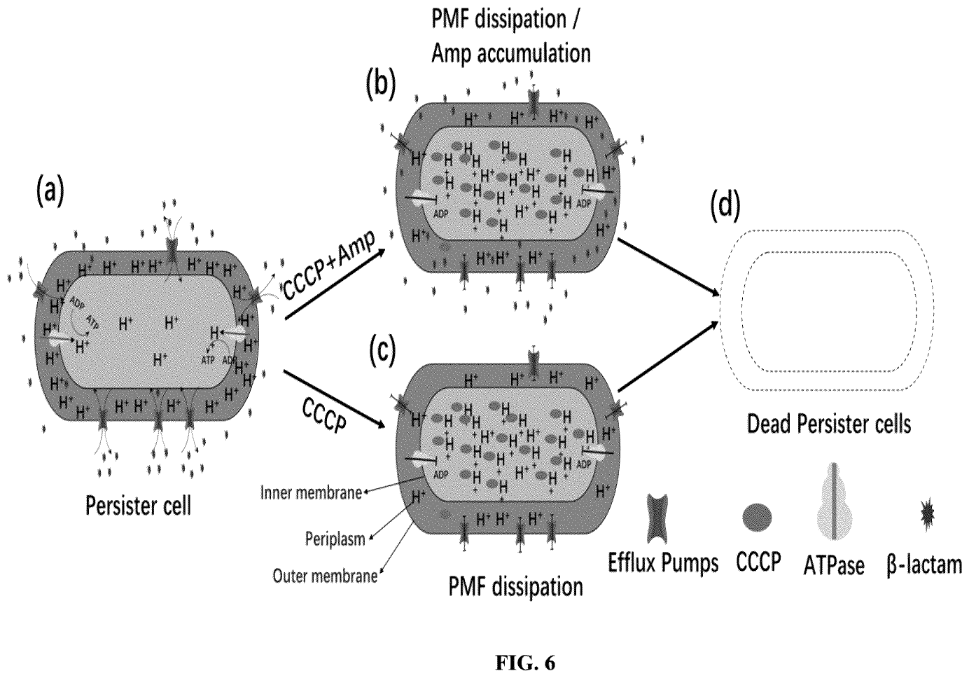

In this work, the reason why changes in tolerance level over a six-days period were monitored was because it was believed that the effect of lack of PMF maintenance function cannot be observed immediately. In fact, various previous studies showed that disrupting PMF and diminishing ATP level could actually lead to formation of tolerance, presumably by triggering dormancy. There is currently no evidence which suggests that PMF is totally dissipated in tolerant cells; on the contrary, PMF is known to be required for the viability of non-replicating M. tuberculosis, as cell death was observed upon inhibition of activities of the ETC, which is essential for generation of PMF. It was also reported that tolerant cells were eradicated in the presence of compounds which cause dissipation of PMF. Therefore, even though PMF dissipation is reported to decrease ATP level and trigger onset of physiological dormancy in bacteria, PMF remains indispensable for prolonged survival of dormant cells. The tolerance-mediating mechanisms are complicated as several lines of evidence show that dormancy is not sufficient or even essential for tolerant cell formation, as tolerant cells that formed in bacterial population with high respiration activity or actively-dividing cells were identified. Orman, M. et al reported that the size of the tolerant sub-population among bacteria with high respiration activity was actually higher than that in cells with low respiration activity, and that inhibition of ETC or the TCA cycle prevented tolerance formation. The present disclosure provides a comprehensive view on the role of PMF in expression of bacterial antibiotic tolerance phenotype by showing that, although dissipation of PMF could trigger tolerance formation even in the absence of starvation stress, a basal level of PMF is actually required for prolonged survival of bacterial tolerant cells. Hence, a lack of the ability to maintain PMF, as in the case of pspA knockout, results in gradual reduction in the size of antibiotic tolerant sub-population when compared to the wild type strain. Inhibition of the ability to generate PMF by treatment with sodium azide also mildly affected tolerance. Importantly, when the ability to generate and maintain PMF was simultaneously inhibited, by treating the pspA knockout mutant with sodium azide, the tolerance level was found to drop drastically (FIG. 6). These observations therefore confirm that active maintenance of a basal level of PMF is required for expression of phenotypic tolerance during nutrient starvation. Consistently, it was found that knocking out key components of the respiratory ETC (ΔnuoIΔndh), which plays a role in generating PMF, resulted in a dramatic drop in tolerance level. The findings regarding the functional importance of PMF in maintenance of tolerance are also consistent with that of Ma et al. who showed that inhibition of energy production by introducing mutations in the sucB and ubiF genes would affect the tolerance level. Taken together, it is highly likely that a basal level of metabolism is maintained in tolerant cells for ATP production and preservation of PMF, possibly through actively scavenging cellular materials released from dead cells as carbon sources. The gene expression data showed that expression of various membrane-bound transporters was up-regulated upon prolonged starvation (FIG. 11).

This disclosure also demonstrates that PMF maintenance was coupled to efflux activities which were also inducible to enhance bacterial survival fitness during starvation (FIG. 11). These efflux activities are presumably involved in export of intracellular antibiotics or toxic metabolites during starvation or other stresses, reducing the amount of antibiotic accumulated intracellularly and enabling organisms under starvation to become antibiotic tolerant. It was shown that efflux systems would lose the driven energy and exhibit decreased efflux efficiency if PMF collapsed. Consistently, Wu et al. showed that structural defect of the AcrAB-TolC pump was associated with reduced antibiotic tolerance. Pu et al. reported that efflux activities were involved in stationary phase-induced tolerance but the underlying regulatory mechanisms were not elucidated. On the other hand, this previous study also showed that specific efflux pumps conferred antibiotic tolerance, but whether such efflux activities are starvation-inducible was not shown. It was confirmed that the efflux system played a role in maintaining phenotypic drug tolerance under prolonged starvation conditions. The findings therefore help bridge this knowledge gap and have important implications in future exploration of starvation-induced tolerance mechanisms and development of anti-tolerance strategies.

The data confirm that the role of PMF is not limited to supporting efflux activity, as PMF dissipation as a result of treatment with CCCP alone leads to rapid eradication of tolerant cells, whereas deletion of efflux genes or treatment with efflux pump inhibitor only resulted in moderate reduction in the size of tolerance population (FIG. 6). PMF is essential for proper functioning of a wide range of membrane proteins, including the aforementioned nutrient scavenging transportation proteins; PMF-dependent mechanisms underlying maintenance of tolerance phenotype remain to be identified. Nevertheless, due to its functional importance in maintaining viability of tolerant cells, PMF was considered as an excellent target for eradication of tolerant cells. Complete eradication of tolerant cells can rarely be achieved by inhibiting one specific cellular function. There were two previous reports of complete eradication of antibiotic tolerant cells in Gram positive bacteria, which involved the use of the retinoid and acyldepsipeptide antibiotic to inflict membrane damage and activate casein lytic proteases respectively. However, these antibiotics are not effective on Gram negative organisms. Disrupting bacterial PMF to completely eradicate tolerant cells of both Gram positive and negative bacteria possesses the clinical significance.

To summarize, this disclosure shows that PMF is essential for prolonged expression of starvation induced antibiotic tolerance phenotype in both Gram-positive and negative bacteria. Findings in this work represent significant advancement in understanding the cellular basis of the phenomenon of bacterial antibiotic tolerance: emergence of antibiotic tolerant population is due to the combined effects of metabolic shutdown and activation of a range of PMF-dependent defense mechanisms in response to variation in environmental conditions, with the latter being particularly important for long-term maintenance of the tolerance phenotype. Inducing dissipation of bacterial PMF could be an effective approach to eradicate bacterial persisters.

An FDA-approved antifungal drug, econazole was identified that can cause dissipation of bacterial PMF and effectively eradicate tolerant cells of S. aureus when used alone, and tolerant cells of Gram negative bacterial pathogens when used in combination with various conventional antibiotics. The combined usage of econazole and ceftazidime was further shown to effectively eradicate bacterial tolerant cells in animal infection models. The findings are highly clinically relevant as they imply that most bacterial species in the tolerance status can be eradicated by PMF-suppressing agents alone or by combined usage of such agents and conventional antibiotics within a 4-days treatment course.

Due to its functional importance in maintaining viability of tolerant cells, PMF is considered as an excellent target for screening of compounds that can eradicate bacterial tolerant sub-population. Complete eradication of tolerant cells is rarely achieved by inhibiting only one specific cellular function. Nevertheless, targeting PMF is increasingly being regarded as a novel antimicrobial strategy. However, most of the PMF dissipators discovered so far, such as CCCP, exhibit high toxicity to human. In this work it was discovered that the imidazole type of antifungal drugs, which are FDA approved drugs, have strong PMF dissipating activity. It is possible that there are other low toxicity compounds that could cause dissipation of bacterial PMF. Attempts should be made to further screen FDA-approved drugs and novel drug leads to identify compounds that can cause dissipation of bacterial PMF without exhibiting toxicity in human. Low toxicity PMF dissipators should be potential good candidates for development of drugs that can kill bacterial persisters.

To summarize, our study identified an FDA-approved antifungal drug, econazole, which could cause dissipation of bacterial PMF. Our findings show that eradication of bacterial tolerant cells of both Gram positive and negative bacteria by a non-toxic PMF-disrupting agent is highly feasible.

Strains and culture. All knockout strains were derived from Escherichia coli BW25113 and single knockout strains were obtained from the Coli Genetics Stock Center (USA) (FIG. 14). Double knockout strains were constructed by the lambda red recombination approach, in which the plasmid pKD46 was used for expression of Red recombinase, which comprises the terminator downstream of frp exo; pKD4 was used to express kanamycin resistance, pCP20 was used for the expression of Flp recombinase. Plasmid pBAD18 was used in gene complementation. Luria-Bertani (LB) broth was used for all cultures unless stated otherwise. All the strains were grown at 37° C. with shaking at 250 rpm.

The following strains were also studied in this study work: E. coli BW25113, carbapenem-resistant E. coli (bla_{NDM}-1-bearing E. coli J53), S. aureus ATCC29213, K. pneumoniae ATCC13833, A. baumannii ATCC19606, P. aeruginosa PA01 and S. typhimurium PY01. Luria-Bertani (LB) broth was used for all cultures unless stated otherwise. All test strains were grown at 37° C. with shaking at 250 rmp/min. DiSC₃(5) was purchased from Thermo Fisher.

Tolerance assay. Upon reaching the exponential phase, bacteria were washed and re-suspended in saline (0.9% NaCl), incubated at 37° C. under constant shaking (250 rpm) for 24 hrs, followed by treatment with ampicillin at a concentration of ˜10×MIC (FIG. 12,3) for 144 hrs (6 days), supplementing fresh ampicillin every 48 hrs. Standard serial dilution and plating on LB agar was performed before and after ampicillin treatment for 4 hrs, 2 days, 4 days and 6 days to determine the fraction of the test population that survived at different time points during the course of treatment.

RNA Sequencing and analysis. Fresh E. coli K-12 BW25113 colonies were inoculated into LB medium and grown overnight at 37° C. under constant shaking (250 rpm). The overnight culture was diluted 100-fold in LB broth and cultivated for about 1 hr until the OD₆₀₀ value reached 0.2 (exponential phase). Aliquots of this exponential phase culture were washed and re-suspended in saline, cultured at 37° C. under constant shaking (250 rpm), followed by incubation with 100 μg/mL ampicillin at 37° C. for 24 hrs. Total RNA of bacteria collected from the exponential phase and starvation phase was extracted by the RNeasy Mini Kit (Qiagen, Germany); rRNA was removed by using the Illumina Ribo-Zero Plus rRNA Depletion Kit; samples was sent to Beijing Genomics Institute (Hong Kong) for transcriptome sequencing. Raw reads were first mapped to the reference genome with Hisat2. These mapped reads were provided as input to Cufflinks, which produced one file of assembled transcripts for each sample. The assembly files were merged with the reference transcriptome annotation into a unified annotation by Cuffmerge, which was quantified by Cuffdiff to generate a set of expression data. Cuffdiff found reads that mapped uniquely to one isoform and calculated isoform abundances, fold changes and q-values. The normalization strategy used was RPKM (Reads Per Kilobase Million) and only the genes whose RPKM was above 5 were chosen to analysis.

Western blot analysis. Upon starvation for 24 hrs, bacteria were harvested by centrifugation and solubilized in sample buffer for 10 mins at 100° C. Total cellular proteins were separated by SDS-PAGE and electroblotted onto PVDF membrane (BIO-RAD 0.2 μM) using a semi-dry electroblotting apparatus (BIO-RAD). Membranes containing fractionated samples were first probed with anti-PspA (polyclonal rabbit source) or anti-GAPDH (Abcam) antibodies and then washed with tris-buffered saline and Tween 20 (TBST). Washed membranes were re-blocked and probed with anti-rabbit antibodies simultaneously. Target protein bands were detected by measurement of chemiluminescence exhibited by the HRP substrate (EMD Millipore); relative band intensities of Western blots were calculated by ImageJ v1.29.

Membrane permeability assay. The membrane permeability or integrity of the test organisms was measured using SYTOX Green (ThermoFisher), which can enter the cell through damaged cell membrane and bind to nucleic acid, generating fluorescence signal. E. coli BW25113 and its ΔpspA derivative at a concentration of OD₆₀₀ of 0.2, which had been subjected to 24 hrs starvation, were collected by centrifugation (6000×g, 2 mins), washed twice and re-suspended in saline. SYTOX Green was then added to give a final concentration of 1 μM, followed by incubation for 30 min in the dark at room temperature. The relative fluorescence signal in the wild type and ΔpspA strain was measured by a Cary Eclipse Fluorescence Spectrophotometer (Agilent), with an excitation wavelength of 488±10 nm and an emission wavelength of 523±10 nm.

Assessment of effect of PAβN on bacterial growth rate. The overnight culture of the Escherichia coli BW25113 strain was diluted 1:100 in LB Broth, followed by addition of 100 μM PAβN; a sample in which only saline was added was included as negative control. OD₆₀₀ value was tested at different time points.

Membrane potential assay. The transmembrane electrical potential was measured by using a membrane potential-sensitive probe, DiSC₃(5). Bacterial population in either the exponential phase (OD₆₀₀ of 0.2) or under 24 hrs starvation (resuspended in saline) were collected by centrifugation (6000×g, 2 mins), washed twice and re-suspended in PBS (pH 7.4), and then adjusted to OD₆₀₀ of 0.2. KCl and DiSC₃(5) were added until final concentration of 100 mM and 1 μM was respectively reached, followed by incubation at room temperature for 25 mins in the dark to allow the dye to penetrate through the outer membrane and produce a quenching effect. Valinomycin (1 μM) was then added to the positive control group to transport K⁺ into cytoplasm, which resulted in depolarization. The fluorescence reading was monitored by using a Clariostar Microplate Reader (BMG LABTECH) at an excitation wavelength of 622±10 nm and an emission wavelength of 670±10 nm for 10 mins. Upon depolarization, the dye was rapidly released into the medium, resulting in dequenching and facilitating detection fluorometrically. Confocal imaging was also conducted for testing the difference between the membrane electrical potential of the wild type strain and the ΔpspA mutant. The sample preparation method is same as that prior to testing with the Microplate Reader except for the last step. Briefly, cells were washed with PBS three times before confocal observation to remove extracellular DiSC₃(5) dye. Bacteria were imaged by the Leica TCS SP8 MP Multiphoton Microscope with a 60× oil-immersion objective. DiSC₃(5) was excited by 638 nm laser and fluorescence was detected by HyD detector at emission wavelength 675±25 nm. The images were acquired and analyzed by the Leica Application Suite X (LAS X) software.

Assessment of effect of proton ionophore and sodium azide on starvation-induced tolerance. To determine whether keeping a significant level of PMF is essential for maintaining a tolerance phenotype in starvation-induced tolerant cells, the effect of the uncoupling agent CCCP or sodium azide (5 mM) was each individually tested. The test agents were added to bacteria which had been subjected to starvation for 24 hrs, followed by incubation at 37° C. and treatment with ˜-10×MIC ampicillin for 144 hrs. Standard serial dilution and plating on LB agar was performed on samples collected every 24 hrs to assess changes in the size of the sub-population that survived during the treatment process. For each sample, a control which did not receive ampicillin treatment was included in the experiment.

Antibiotic accumulation assay. The overnight bacteria culture was diluted 100-fold in LB broth and cultivated for about 1 hr until the OD₆₀₀ value reached 0.2 (exponential phase). Aliquots of this exponential phase culture were washed and re-suspended in saline, cultured at 37° C. under constant shaking (250 rpm) for 24 hrs, followed by addition of CCCP (1 μM). After 5 mins, BOCILLIN™ FL Penicillin (10 μg/mL) was added and incubated at 37° C. with shaking at 250 rpm for 1 hr. Upon washing twice with PBS, fluorescence signal was measured by flow cytometry CytoFLEX (Beckman). Microorganisms were identified by FSC (forward scatter) and SSC (side scatter) parameters. Fluorescence intensity was measured at 488-nm excitation, 525-nm emission.

Assessment of efflux activity. A10-mL portion of bacterial population which had been subjected to 24 hrs starvation was centrifuged at 6000×g for 5 mins at room temperature. The pellet was re-suspended in PBS containing 1 mM MgCl₂(PPB) and adjusted to OD₆₀₀ 0.2. Nile Red which fluoresces only weakly in aqueous solutions but becomes strongly fluorescent in nonpolar environments was added to produce a final concentration of 5 μM followed by incubation at 37° C. for 30 mins, with 250 rpm shaking. CCCP was then added to produce a final concentration of 100 μM; fluorescence was measured for a period of 30 mins by a Clariostar Microplate Reader at an excitation wavelength of 544±10 nm and an emission wavelength of 650±10 nm.

Determination of minimal inhibitory concentrations (MICs). The MIC of ampicillin against A. baumannii ATCC19606, K. pneumoniae ATCC13883, P aeruginosa PAO1, S. aureus ATCC29213, S. typhimurium PY01 and E. coli K-12 BW25113 and its gene knockout derivatives (obtained from the Keio collection) was determined by incubating freshly grown cultures (Mueller Hinton Broth (MHB) (BD Difco, America) with various concentrations of ampicillin for 16 hours, recording the minimal concentration that inhibited bacterial growth and resulted in a lack of turbidity. Results were based on the average of at least three independent experiments and interpreted according to CLSI guidelines.

Determination of Minimal Inhibitory Concentrations (MICs). The MIC of ampicillin or econazole against Acinetobacter baumannii strain ATCC19606, Klebsiella pneumoniae ATCC13883, Pseudomonas aeruginosa PAO1, Staphylococcus aureus ATCC29213, S. typhimurium PY01 and E. coli BW25113 was determined by incubating freshly grown cultures (Mueller Hinton Broth (MHB) (BD Difco, America) with various concentrations of ampicillin or econazole for 16 hours, recording the concentration that inhibited bacterial growth and resulted in a lack of turbidity. Results were based on the average of at least three independent experiments and interpreted according to the CLSI guidelines.

### Tolerance Assay

Upon reaching the exponential phase, bacteria were washed and re-suspended in saline (0.9% NaCl), and then incubated at 37° C. under constant shaking (250 rpm/min) for 24 hrs. The bacterial population under starvation was then treated with econazole 40 μM, CCCP 100 μM, meropenem 40 μg/ml, gentamycin 20 μg/ml, ciprofloxacin 1 μg/ml, ceftazidime 100 μg/ml, ampicillin at a concentration of 10×MIC or combing treated with those conventional antibiotics and econazole/CCCP for 96 hrs (4 days). Standard serial dilution and plating on LB agar were performed before and after ampicillin treatment for 4 hrs, 1 day, 2 days and 4 days to determine the fraction of the test population that survived at different time points during the course of treatment.

Assessment of effect of econazole on membrane potential. Fluorometric measurement of membrane potential of bacterial cells was performed using the voltage-sensitive dye DiSC3(5). The test organisms were first subjected to starvation for 24 hrs and then centrifuged and washed twice with PBS. The cell pellet was resuspended in PBS containing 100 mM of KCl to a final concentration of OD 0.2. The cells were incubated with 1 μM of DiSC3(5) for 5 min under shaking in dark. The cells were treated with econazole (40 μM); a no treatment control was included. The fluorometric measurements were carried out on black polystyrene microtiter plates using a Clariostar Microplate Reader (BMG LABTECH), with excitation wavelength at 610 nm and emission wavelength at 660 nm.

Electron microscopy analysis. E. coli which had been subjected to starvation for 24 hours were treated with econazole alone, ampicillin alone and a combination of econazole and ampicillin for 24 hours, followed by examination under scanning electron microscopy (SEM). Cells treated with saline were included as negative control. Briefly, bacterial cells were fixed in 0.4% polyoxymethylene overnight and then in Osmium tetroxide (OsO₄) for 2 hrs, followed by washing for three times with PBS. The cells were then dehydrated using pure ethanol, and infiltrated and embedded in Spurr resin for examination by SEM.

### Mouse Deep-Seated Thigh Infection Model

NIH mice were purchased from the Guangdong Center for Experimental Animals, Guangzhou, China. Male mice at ˜6 weeks of age with a weight of ˜20 g were used in the experiments, 6 mice per group. The animals were allowed to acclimate to the housing facility for 5 days. The mice were made neutropenic by administering 150 mg/kg cyclophosphamide 3 days and 1 day before infection. An inoculum of 1×10⁶ CFU of E. coli BW25113 was injected into the right thigh of the mouse. At 24 hrs post-infection, the mice in each group received ceftazidime (20 mg/kg), econazole (20 mg/kg) or ceftazidime combining econazole treatment (i.p.) every 12 hrs for 72 hrs randomly. The mice were then euthanized and the infected thighs were aseptically excised and homogenized in PBS; the number of E. coli present in the samples was enumerated by serial dilution, spreading on LB plates, and incubation at 37° C. overnight. The population size of bacteria that survived different treatments was recorded, compared and analyzed by one-way ANOVA and post hoc Tukey test. The data were presented by using the Graph Pad Prism software. All experimental protocols followed the standard operating procedures of the Biosafety level 2 animal facilities approved by the Animal Ethics Committee of The City University of Hong Kong.

### Mouse Peritonitis Infection Model

NIH male mice of about six-weeks-old with body weight of ˜20 g were used, 6 mice per group. The animals were allowed to acclimate to the housing facility for 5 days. The mice were made neutropenic by administering 150 mg/kg cyclophosphamide 3 days and 1 day before infection. Different amounts of S. typhimurium strain PY01 (2.8×10⁵ CFU, 7.6×10⁵ CFU or 1.5×10⁶ CFU) were inoculated into the animals via intraperitoneal injection. The mice were subjected to ceftazidime (20 mg/kg), econazole (20 mg/kg) or ceftazidime combining econazole treatment (i.p.) 24 h after inoculation, at 12 hrs intervals for 72 hrs randomly. The mortality rate of the test mice was recorded every 12 hrs. After 72 h of treatment, live mice were euthanized and subjected to peritoneal washes which involved injection of 2 mL of saline into the intraperitoneal space, followed by abdominal massage. The abdomen was then cut open and 200 μL of peritoneal fluid were collected and serially diluted in saline. A 100 μL portion of each dilution was spread on LB plates, followed by incubation overnight at 37° C. Colonies were counted to determine the bacterial load in the test samples, which were expressed as CFU/ml. Statistical analysis and ethic approval were the same as described above.

### Quantification and Statistical Analysis

Statistical methods used in this work are described in the figure legends. Statistical analysis was performed by using the GraphPad Prism software version 7.00 (Prism). The averages are shown, with error bars indicating the SD. Two-tailed Student's t test were used. ns, —not significant; *p<0.05; **p<0.01; ***p<0.001; ****p<0.0001.

What is claimed is:

1. A method for treating a bacterial infection in a subject in need thereof, the method comprising: administering a therapeutically effective amount of a bacterial transmembrane proton motive force (PMF) inhibitor to the subject, wherein the bacterial infection is the result of a bacterial cell population comprising persister bacterial cells, antibiotic resistant bacterial cells, or a mixture thereof, wherein the PMF inhibitor is an imidazole-based antifungal agent with the proviso that the PMF inhibitor is not 4-(2-amino-1H-imidazol-4-yl)-N-(tridecan-7-yl)butanamide or 4-(2-amino-1H-imidazol-4-yl)-N-tridecylbutanamide.

2. The method of claim 1, wherein the bacterial cell population is a Gram-negative bacterial cell population.

3. The method of claim 1, wherein the imidazole-based antifungal agent is selected from the group consisting of clotrimazole, econazole, sertaconazole, sulconazole, tioconazole, luliconazole, isoconazole, miconazole, enilconazole, fenticonazole, ketoconazole, climbazole, butoconazole, oxiconazole, fluconazole, voriconazole, letrozole, triclabendazole, thiabendazole, fenbendazole, and omeprazole or a pharmaceutically acceptable salt thereof.

4. The method of claim 1, wherein the PMF inhibitor is administered in an amount effective to at least partially inhibit PMF in the bacterial cell population.

5. The method of claim 1, wherein the bacterial infection is the result of a bacterial cell population consisting of 50% or more of persister bacterial cells, antibiotic resistant bacterial cells, or a mixture thereof.

6. The method of claim 1, wherein the bacterial infection is the result of a bacterial cell population consisting of 90% or more of persister bacterial cells, antibiotic resistant bacterial cells, or a mixture thereof.

7. The method of claim 1, wherein the bacterial infection is the result of a bacterial cell population consisting essentially of antibiotic resistant bacteria selected from the group consisting of E. coli, K. pneumoniae, A. baumannii, P. aeruginosa, S. aureus, and S. typhimurium.

8. The method of claim 1 further comprising the step of co-administering a therapeutically effective amount of an antibacterial to the subject.

9. The method of claim 8, wherein the bacterial infection is the result of a bacterial cell population consisting of 50% or more of persister bacterial cells, antibiotic resistant bacterial cells, or a mixture thereof.

10. The method of claim 8, wherein the imidazole-based antifungal agent is selected from the group consisting of econazole, sertaconazole, sulconazole, tioconazole, luliconazole, and isoconazole, or a pharmaceutically acceptable salt thereof.

11. The method of claim 8, wherein the antibacterial is selected from the group consisting of: a β-lactam, an aminoglycoside, a quinolone, a glycopeptide, a glycylcycline, a lipopeptide, a macrolide, chloramphenicol, a dihydrofolate reductase inhibitor, a sulfonamide, rifampicin, metronidazole, clindamycin, linkomycin, fusidic acid, furazolidone, isoniazid, and pyrazinamide.

12. The method of claim 8, wherein the antibacterial is selected from the group consisting of ampicillin, ceftazidime, ciprofloxacin, gentamycin, meropenem, and colistin or a pharmaceutically acceptable salt thereof.

13. The method of claim 8, wherein the imidazole-based antifungal agent is selected from the group consisting of econazole, sertaconazole, sulconazole, tioconazole, luliconazole, isoconazole, and miconazole or a pharmaceutically acceptable salt thereof; and the antibacterial is colistin or a pharmaceutically acceptable salt thereof.

14. The method of claim 8, wherein the imidazole-based antifungal agent is econazole or a pharmaceutically acceptable salt thereof; and the antibacterial is selected from the group consisting of ampicillin, ceftazidime, ciprofloxacin, gentamycin, meropenem, and colistin or a pharmaceutically acceptable salt thereof.

15. The method of claim 14, wherein the bacterial infection is the result of a bacterial cell population consisting of 90% or more of persister bacterial cells, antibiotic resistant bacterial cells, or a mixture thereof.

16. A method of re-sensitizing a persister bacterial cell or an antibiotic resistant bacterial cell to an antibacterial, the method comprising: contacting the persister bacterial cell or the antibiotic resistant bacterial cell with a PMF inhibitor, wherein the PMF inhibitor is an imidazole-based antifungal agent with the proviso that the PMF inhibitor is not 4-(2-amino-1H-imidazol-4-yl)-N-(tridecan-7-yl)butanamide or 4-(2-amino-1H-imidazol-4-yl)-N-tridecylbutanamide.

17. The method of claim 16, wherein the persister bacterial cell or the antibiotic resistant bacterial cell is a Gram-negative persister bacterial cell or Gram-negative antibiotic resistant bacterial cell.

18. The method of claim 16, wherein the imidazole-based antifungal agent is selected from the group consisting of clotrimazole, econazole, sertaconazole, sulconazole, tioconazole, luliconazole, isoconazole, miconazole, enilonazole, fenticonazole, ketoconazole, climbazole, butoconazole, oxiconazole, fluconazole, voriconazole, letrozole, triclabendazole, thiabendazole, fenbendazole, and omeprazole or a pharmaceutically acceptable salt thereof.

19. The method of claim 16, wherein the imidazole-based antifungal agent is econazole or a pharmaceutically acceptable salt thereof.

20. The method of claim 16, wherein the persister bacterial cell or the antibiotic resistant bacterial cell is selected from the group consisting of E. coli, K. pneumoniae, A. baumannii, P. aeruginosa, S. aureus, and S. typhimurium.
